(19) United States
(12) Patent Application Publication
(10) Pub. No.: US 2010/0114236 A1
(43) Pub. Date: May 6, 2010

(54) HYBRID BATTERY SYSTEM WITH BIOELECTRIC CELL FOR IMPLANTABLE CARDIAC THERAPY DEVICE

(75) Inventors: Naixiong Jiang, Mountain View, CA (US); Gene A. Bornzin, Simi Valley, CA (US); John W. Poore, South Pasadena, CA (US)

Correspondence Address: STEVEN M MITCHELL;PACESETTER INC, 701 EAST EVELYN AVENUE, SUNNYVALE, CA, 94086, US

(73) Assignee: PACESETTER INC., Sunnyvale, CA (US)

(21) Appl. No.: 12/263,348

(22) Filed: Oct. 31, 2008

(51) Int. Cl. A61N 1/362; H01M 10/36

(52) U.S. Cl. 607/35; 429/2

(57) ABSTRACT

A system and method for powering an implantable cardiac therapy device (ICTD) via a hybrid battery system. The hybrid battery is comprised of a low voltage and low current bioelectric cell, a high voltage and high current rechargeable cell, and a charging means. Via the charging means, the bioelectric cell maintains the rechargeable cell at or near full power. The rechargeable cell is configured to power some or all operations of the ICTD. Some ICTD operations may be powered directly by the bioelectric cell. The rechargeable cell is further configured to be charged via a continuous charging process, reducing the complexity of the charging circuitry. In an embodiment, at least the bioelectric cell is external to the ICTD, enabling easy replacement of this power source. In an embodiment, a consumable anode of the bioelectric cell is external to the ICTD, enabling replacement of the power source by replacing only the anode.

### RELATED APPLICATIONS

This application is related to co-pending and commonly-owned U.S. patent application Ser. No. 11/737,307, entitled “Bioelectric Battery for Implantable Device Applications”, filed Apr. 19, 2007; co-pending and commonly-owned U.S. patent application Ser. No. ______, entitled “Hybrid Battery System For Implantable Cardiac Therapy Device”, filed on even date herewith (attorney docket number A06E3099); and co-pending and commonly-owned U.S. patent application Ser. No. 11/940,552, entitled “Blood Oxygen Saturation Measurement Utilizing A Bioelectric Battery”, filed Nov. 15, 2007; each of which is incorporated by reference herein as if reproduced in full below.

### BACKGROUND

1. Field of the Invention

The present invention relates generally to implantable cardiac therapy devices, and to power sources for the same. More particularly, the invention relates to a hybrid battery system which includes a bioelectric cell coupled to a rechargeable cell.

2. Background Art

Implantable cardiac therapy devices (ICTDs) enjoy widespread use for providing convenient, portable, sustained therapy for cardiac patients with a variety of cardiac arrhythmias. An ICTD may be a pacemaker, or may combine a pacemaker and defibrillator in a single implantable device. Such devices may be configured to provide ongoing cardiac pacing in order to maintain an appropriate cardiac rhythm. In addition, should the ICTD detect that the patient is experiencing an episode of ventricular fibrillation (or an episode of ventricular tachycardia), an ICTD may be configured to deliver appropriate defibrillation therapy.

An ICTD requires a portable power supply in the form of a battery. The battery has several inherent requirements including safety and also the ability to provide power to the ICTD for an extended period of time, thereby minimizing the frequency of invasive procedures to replace the battery.

ICTDs have additional, specialized power requirements due to the specific nature of their function. Long-term cardiac pacing can be supported by a low voltage, low current power source. Defibrillation therapy, however, requires rapid, high voltage, high current delivery to the heart.

A standard battery for many ICTD applications has been the Lithium Silver Vanadium Oxide (LiSVO) cell, which provides sufficient voltage for cardiac pacing and background operations such as sensing and communications. The LiSVO cell also can provide an adequate, if not entirely ideal, voltage and current flow for cardiac shocking (that is, defibrillation therapy).

However, the LiSVO battery suffers from disadvantages as well. Its internal resistances from both the anode and cathode tend to increase in the discharging process, particularly during midlife. As a result, over time, the loaded voltage will be lower and the time for discharging (that is, the time for charging the shocking capacitors of the ICTD) increases. In some cases, the discharge time could be doubled, which may render the battery unacceptable for defibrillation. This may result in a medical decision to replace the device, which in turn means the patient may have to accept a premature surgery. In the past, the increased battery discharge time has been a major issue for ICTDs.

Another possible power source for an ICTD is a bioelectric battery, which generates energy from a replenishable substance of the patient. Typically, a body fluid is an electrolyte, providing the replenishable substance. In one embodiment the replenishable substance may be the blood oxygen of the patient. Embodiments of such a bioelectric battery are described, for example, in co-pending and commonly-owned U.S. patent application Ser. No. 11/737,307, entitled “Bioelectric Battery for Implantable Device Applications”, filed Apr. 19, 2007, which is incorporated by reference herein in its entirety.

An implantable bioelectric battery configured to generate power from a replenishable substance of the patient may present significant advantages as a power source, as compared to standard power cells (such as the LiSVO power cells currently employed in many ICTDs). For example, a bioelectric cell may have a more consistent current and/or voltage delivery over the lifetime of the cell. In addition, the bioelectric cell may also have a longer lifetime than the LiSVO cell, and therefore require less frequent replacement. This spares the patient unnecessary surgery.

However, a bioelectric power cell may not offer all the features or power capabilities desired for an ICTD. For example, the relatively low current available from a bioelectric cell (e.g., on the order of 100 μAmps) may not be sufficient to power high voltage shocking. Also, the current available from a bioelectric cell may not be sufficient for certain kinds of data telemetry, or for certain high speed telemetry data rates. Further, because the bioelectric cell requires a replenishable substance of a patient to generate power, the cell cannot provide any power when the device is not implanted in a patient. However, power may be required, even during non-implantation, for device testing, final programming, and during the pre-implant shelf life of the ICTD.

In short, there does not exist a single power cell which is optimized to effectively provide optimized electrical sourcing for an ICTD.

A recent improvement has been the use of a hybrid battery source. A hybrid battery system combines two different physical batteries, with different but complementary electrical properties, into a single functional package. The single functional package effectively serves as the battery for the ICTD. A first physical battery (which may also be referred to as a cell) of the hybrid battery typically has a high energy density for long battery life, but may have a relatively low voltage and low current output. A second physical battery (or cell) has higher voltage output, higher current delivery (typically a result of lower internal resistance), and superior recharging time and recharging properties. However, the second cell typically has lower energy density that the first battery. The two cells are coupled in the hybrid battery, with the first cell providing charging to the second cell.

In many applications, a hybrid battery may be an improvement over the standard batteries (such as the Li/SVO cells) currently employed in many ICTD applications. However, the lifetime of a hybrid battery is still limited by the energy storage of the primary cell.

What is needed, then, is a battery designed for use in an ICTD which takes advantage of the optimized electrical properties of a hybrid battery design, and which further takes advantage of long life and other benefits of a bioelectric cell.

### BRIEF SUMMARY

The present system and method employs a hybrid battery comprised of at least two types of cells to power an implantable cardiac therapy device (ICTD). A first type of cell is a bioelectric cell which generates electrical power from a replenishable organic substance. In one example embodiment, the bioelectric cell provides low voltage but high energy density.

The bioelectric cell is coupled to a secondary cell which is not a bioelectric cell. The bioelectric cell is coupled to the secondary cell via charging means, which may for example be a simple DC-to-DC converter. The secondary cell is maintained at full or nearly full charge by the energy provided by the bioelectric cell. In one example embodiment, the secondary cell has low internal resistance and high voltage, making it suitable to rapidly charge ICTD capacitors for cardiac shocking (e.g., for defibrillation). The secondary cell may also provide power for other ICTD operations which may require relatively high voltage or high current. For example, the secondary cell may power high speed data telemetry.

In an embodiment, the bioelectric cell directly provides power to the ICTD for purposes of routine cardiac monitoring, pacing, and other low voltage, low current operations. In an alternative embodiment, the secondary cell provides power to the ICTD for purposes of routine cardiac monitoring, pacing, and other low voltage, low current operations. In another alternative embodiment, some low current operations (cardiac monitoring, pacing, etc.) are powered via power from the bioelectric cell, while other low current operations are powered via power from the secondary cell.

An optimized energy density distribution may be implemented between the bioelectric cell and the secondary cell. In one embodiment, the first type of cell is the bioelectric cell, while the secondary cell is a Li ion polymer cell. Each type of cell may be implemented as a single physical cell, or alternatively as two or more physical cells of the same type.

Further embodiments, features, and advantages of the present system and method, as well as the structure and operation of various exemplary embodiments of the present system and method, are described in detail below with reference to the accompanying drawings.

### BRIEF DESCRIPTION OF THE DRAWINGS/FIGURES

The accompanying drawings, which are incorporated herein and form part of the specification, illustrate the methods and systems presented herein for a hybrid bioelectric battery optimized for an implantable cardiac therapy device. Together with the detailed description, the drawings further serve to explain the principles of and to enable a person skilled in the relevant art(s) to make and use the methods and systems presented herein.

In the drawings, like reference numbers indicate identical or functionally similar elements. Further, the drawing in which an element first appears is typically indicated by the leftmost digit(s) in the corresponding reference number (e.g., an element numbered 302 first appears in FIG. 3). (There are some exceptions. For example, element 404 first appears in FIG. 2B of this document, but is discussed in detail in conjunction with FIG. 4.)

Additionally, some elements may be labeled with only a number to indicate a generic form of the element, while other elements labeled with the same number followed by another number or a letter (or a letter/number combination) may indicate a species of the element. A period or underscore may be introduced in the label for clarity of reading, and has no other significance.

FIG. 1 is a simplified diagram illustrating an exemplary implantable cardiac therapy device (ICTD) having a bioelectric cell and being in electrical communication with a patient's heart by means of leads suitable for delivering multi-chamber stimulation and pacing therapy, and for detecting cardiac electrical activity.

FIG. 2A is a functional block diagram of an exemplary ICTD that can detect cardiac electrical activity and analyze cardiac electrical activity, as well as provide cardioversion, defibrillation, and pacing stimulation in four chambers of a heart.

FIG. 2B is a functional block diagram of another exemplary ICTD with an exemplary hybrid battery which includes a bioelectric cell.

FIG. 2C is a functional block diagram of another embodiment of an exemplary ICTD with an exemplary hybrid battery which includes a bioelectric cell.

FIG. 3 is a functional block diagram of the internal architecture and principle external connections of an exemplary external programming device which may be used by to monitor or program an ICTD.

FIG. 4A-4D show functional block diagrams of exemplary bioelectric hybrid battery systems employing a bioelectric cell, along with interconnections to some elements of an exemplary ICTD, according to embodiments of the present system and method.

FIG. 5 shows a set of experimentally measured plots of the time required for various Li ion polymer cells to charge shocking capacitors in a representative ICTD.

FIG. 6 shows a set of experimentally measured plots of the time required for various Li ion polymer cells to charge shocking capacitors in another representative ICTD.

FIG. 7 shows a set of experimentally measured plots of the time required for a representative Li ion polymer cell to charge the shocking capacitors of a representative ICTD at different current levels.

### DETAILED DESCRIPTION

### 1. Overview

### 2. Exemplary Environment—Overview

3. Exemplary ICTD in Electrical Communication with a Patient's Heart

### 4. Exemplary Bioelectric Cell

### 5. Functional Elements of an Exemplary ICTD

### 6. ICTD Programmer

7. Hybrid Battery with Bioelectric Cell
8. Further Elements of Hybrid Battery with Bioelectric Cell

### 9. Choice of Secondary Power Cell

10. Lithium Ion Polymer Cell vs. Standard Lithium Ion Cell

### 11. Storage Capacities and Power Delivery for Cells for Different ICTD Applications

### 12. Alternative Embodiments

### 13. Conclusion

### 1. Overview

The following detailed description of systems and methods for a hybrid battery system with a bioelectric cell for implantable cardiac therapy devices refers to the accompanying drawings that illustrate exemplary embodiments consistent with these systems and methods. Other embodiments are possible, and modifications may be made to the embodiments within the spirit and scope of the methods and systems presented herein. Therefore, the following detailed description is not meant to limit the methods and systems described herein. Rather, the scope of these methods and systems is defined by the appended claims.

It would be apparent to one of skill in the art that the systems and methods for a hybrid battery system with a bioelectric cell for implantable cardiac therapy devices, as described below, may be implemented in many different embodiments of hardware, software, firmware, and/or the entities illustrated in the figures. Any actual hardware and/or software described herein is not limiting of these methods and systems. In addition, more than one embodiment of the present system and method may be presented below, and it will be understood that not all embodiments necessarily exhibit all elements, that some elements may be combined or connected in a manner different than that specifically described herein, and that some differing elements from the different embodiments presented herein may be functionally and structurally combined to achieve still further embodiments of the present system and method.

Thus, the operation and behavior of the methods and systems will be described with the understanding that modifications and variations of the embodiments are possible, given the level of detail presented herein.

### 2. Exemplary Environment—Overview

Before describing in detail the methods and systems for a hybrid battery system with a bioelectric cell for implantable cardiac therapy devices, it is helpful to describe an example environment in which these methods and systems may be implemented. The methods and systems described herein may be particularly useful in the environment of an implantable cardiac therapy device (ICTD).

An ICTD may also be referred to synonymously herein as a “stimulation device”, emphasizing the role of the ICTD in providing pacing and shocking to a human heart. However, an ICTD may provide operations or services in addition to stimulation, including but not limited to cardiac monitoring. Also, some ICTDs may provide cardiac pacing and monitoring, but may not provide cardiac shocking (that is, may not provide defibrillation).

The bioelectric hybrid battery described herein, as well as the ICTD described herein, are typically implanted in a living organism which is typically a mammal, and is typically a human being, though these devices may be implanted in other mammals as well. The human being is typically referred to as a patient. The terms “organism”, “mammal”, “person”, and “patient” may be used interchangeably in this document to refer to the organism in which an ICTD may be implanted, and in which a bioelectric hybrid battery may be implanted.

An ICTD is a physiologic measuring device and therapeutic device that is implanted in a patient to monitor cardiac function and to deliver appropriate electrical therapy, for example, pacing pulses, cardioverting and defibrillator pulses, and drug therapy, as required. ICTDs include, for example and without limitation, pacemakers, cardioverters, defibrillators, implantable cardioverter defibrillators, implantable cardiac rhythm management devices, and the like. Such devices may also be used in particular to monitor cardiac electrical activity and to analyze cardiac electrical activity. The term “implantable cardiac therapy device” or simply “ICTD” is used herein to refer to any such implantable cardiac therapy device.

Persons skilled in the relevant arts will recognize that the term “battery” is sometimes employed in place of the word “cell” so that, for example, a “lithium ion polymer cell” may also be described, equivalently, as a “lithium ion polymer battery”. Similarly, the terms “bioelectric cell” and “bioelectric battery” may be used interchangeably within the art.

Within this document, individual batteries (a bioelectric battery, a standard lithium ion battery, a lithium ion polymer battery, a lithium/silver vanadium oxide battery, a lithium magnesium oxide battery, etc.) are generally referred to as “cells” rather than batteries. So, for example, the usage is “a bioelectric cell”, “a lithium ion polymer cell”, etc. This usage is strictly to help distinguish these cells from the overall hybrid battery system of the present system and method. The hybrid battery system of the present system and method is comprised of multiple cells. The usage employed herein (“cell” for individual batteries vs. “battery” for the hybrid battery system) has no further significance.

### 3. Exemplary ICTD in Electrical Communication with a Patient's Heart

The techniques described below are intended to be implemented in connection with any ICTD or any similar stimulation device that is configured or configurable to stimulate nerves and/or stimulate and/or shock a patient's heart.

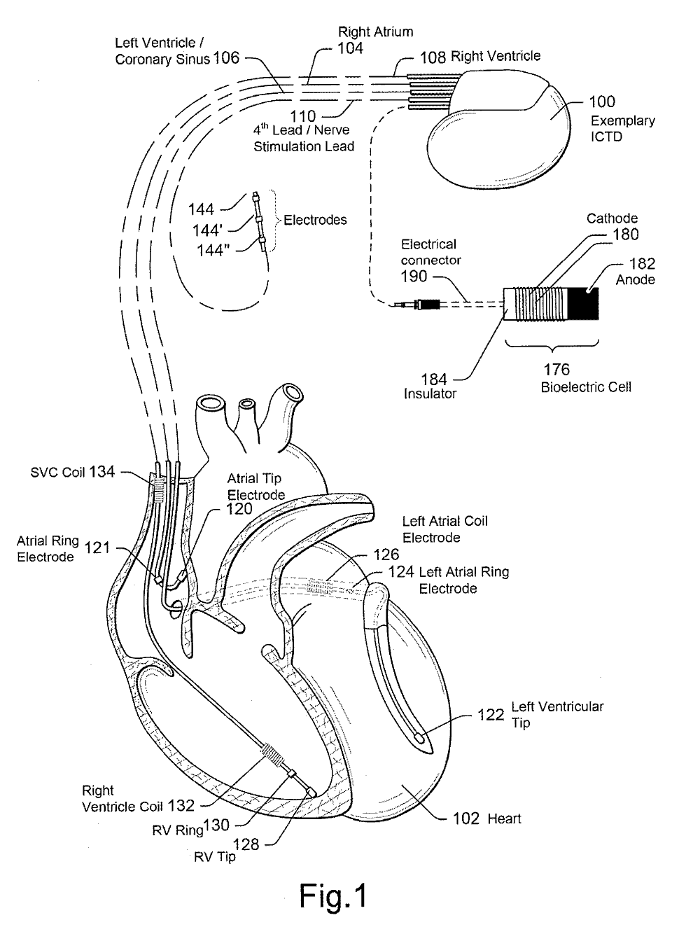

FIG. 1 shows an exemplary stimulation device or ICTD 100 in electrical communication with a patient's heart 102 by way of three leads 104, 106, 108, suitable for delivering multi-chamber stimulation and shock therapy. The leads 104, 106, 108 are optionally configurable for delivery of stimulation pulses suitable for stimulation of autonomic nerves. In addition, the device 100 includes a fourth lead 110 having, in this implementation, three electrodes 144, 144′, 144″ suitable for stimulation of autonomic nerves. This lead may be positioned in and/or near a patient's heart or near an autonomic nerve within a patient's body and remote from the heart. Of course, such a lead may be positioned epicardially or at some other location to stimulate other tissue.

The right atrial lead 104, as the name implies, is positioned in and/or passes through a patient's right atrium. The right atrial lead 104 optionally senses atrial cardiac signals and/or provide right atrial chamber stimulation therapy. As shown in FIG. 1, the stimulation device 100 is coupled to an implantable right atrial lead 104 having, for example, an atrial tip electrode 120, which typically is implanted in the patient's right atrial appendage. The lead 104, as shown in FIG. 1, also includes an atrial ring electrode 121. Of course, the lead 104 may have other electrodes as well. For example, the right atrial lead optionally includes a distal bifurcation having electrodes suitable for stimulation of autonomic nerves.

To sense atrial cardiac signals, ventricular cardiac signals and/or to provide chamber pacing therapy, particularly on the left side of a patient's heart, the stimulation device 100 is coupled to a coronary sinus lead 106 designed for placement in the coronary sinus and/or tributary veins of the coronary sinus. Thus, the coronary sinus lead 106 is optionally suitable for positioning at least one distal electrode adjacent to the left ventricle and/or additional electrode(s) adjacent to the left atrium. In a normal heart, tributary veins of the coronary sinus include, but may not be limited to, the great cardiac vein, the left marginal vein, the left posterior ventricular vein, the middle cardiac vein, and the small cardiac vein.

Accordingly, an exemplary coronary sinus lead 106 is optionally designed to receive atrial and ventricular cardiac signals and to deliver left ventricular pacing therapy using, for example, at least a left ventricular tip electrode 122, left atrial pacing therapy using at least a left atrial ring electrode 124, and shocking therapy using at least a left atrial coil electrode 126. For a complete description of a coronary sinus lead, the reader is directed to U.S. Pat. No. 5,466,254, “Coronary Sinus Lead with Atrial Sensing Capability” (Helland), which is incorporated herein by reference. The coronary sinus lead 106 further optionally includes electrodes for stimulation of autonomic nerves. Such a lead may include pacing and autonomic nerve stimulation functionality and may further include bifurcations or legs. For example, an exemplary coronary sinus lead includes pacing electrodes capable of delivering pacing pulses to a patient's left ventricle and at least one electrode capable of stimulating an autonomic nerve. An exemplary coronary sinus lead (or left ventricular lead or left atrial lead) may also include at least one electrode capable of stimulating an autonomic nerve, such an electrode may be positioned on the lead or a bifurcation or leg of the lead.

Stimulation device 100 is also shown in electrical communication with the patient's heart 102 by way of an implantable right ventricular lead 108 having, in this exemplary implementation, a right ventricular tip electrode 128, a right ventricular ring electrode 130, a right ventricular (RV) coil electrode 132, and an SVC coil electrode 134. Typically, the right ventricular lead 108 is transvenously inserted into the heart 102 to place the right ventricular tip electrode 128 in the right ventricular apex so that the RV coil electrode 132 will be positioned in the right ventricle and the SVC coil electrode 134 will be positioned in the superior vena cava. Accordingly, the right ventricular lead 108 is capable of sensing or receiving cardiac signals, and delivering stimulation in the form of pacing and shock therapy to the right ventricle. An exemplary right ventricular lead may also include at least one electrode capable of stimulating an autonomic nerve, such an electrode may be positioned on the lead or a bifurcation or leg of the lead.

### 4. Exemplary Bioelectric Cell

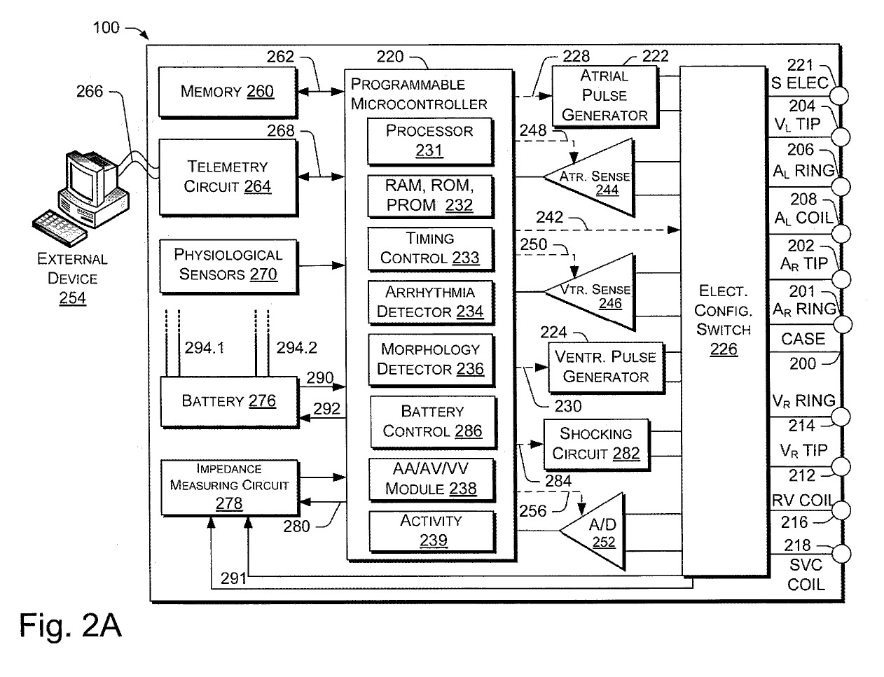

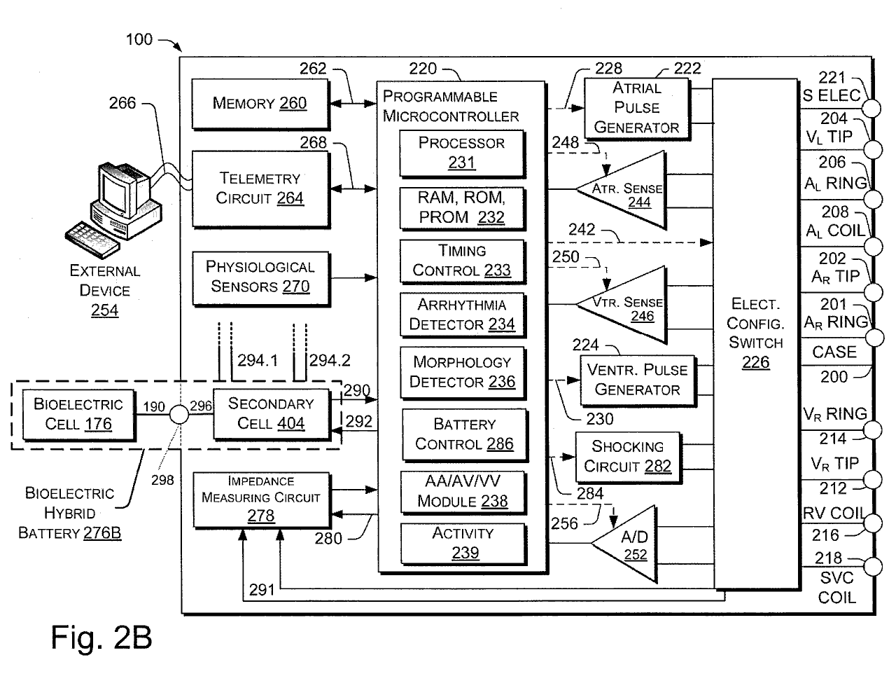

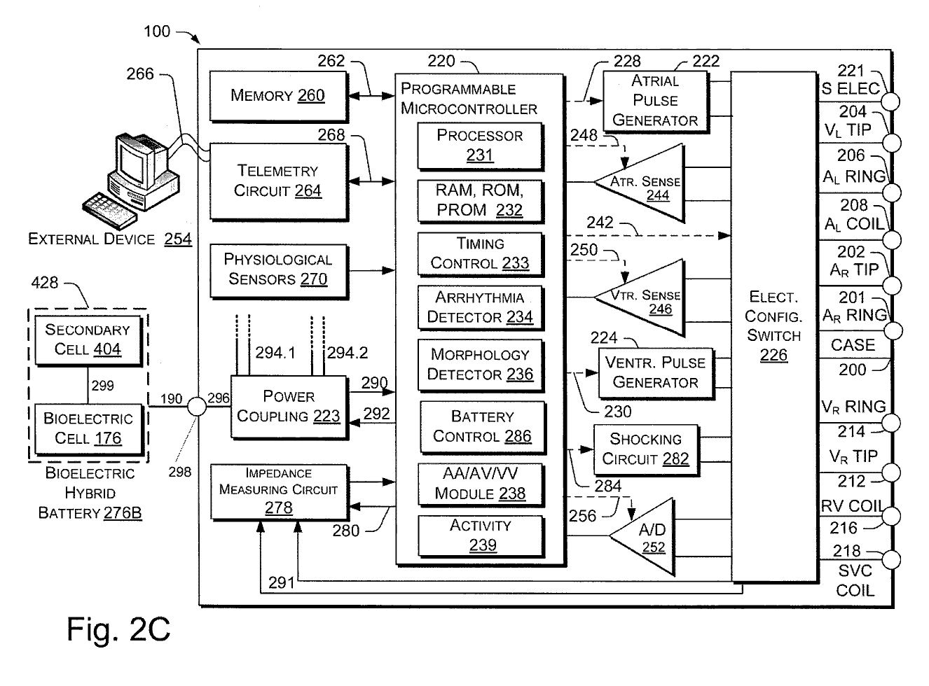

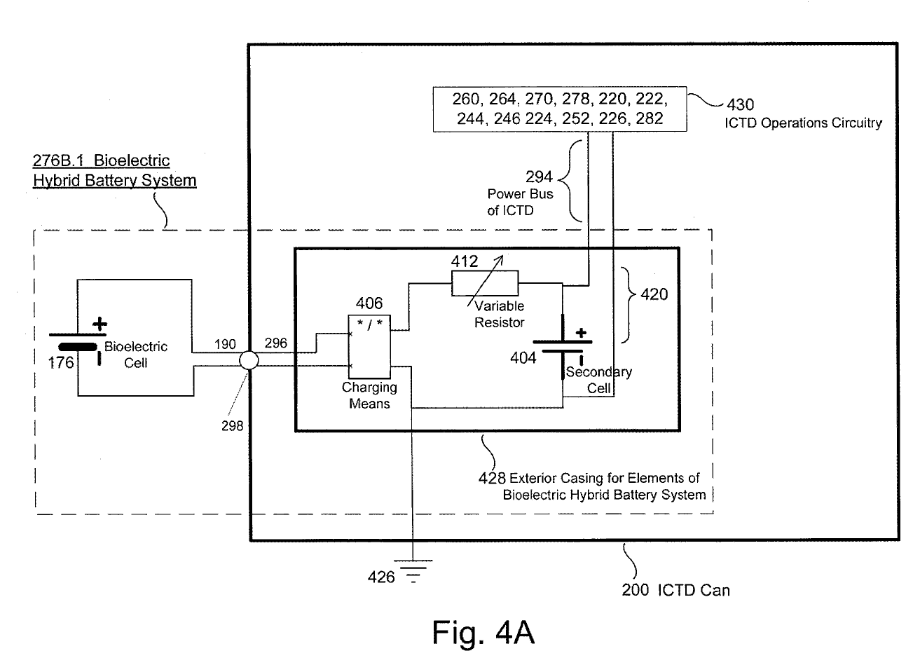

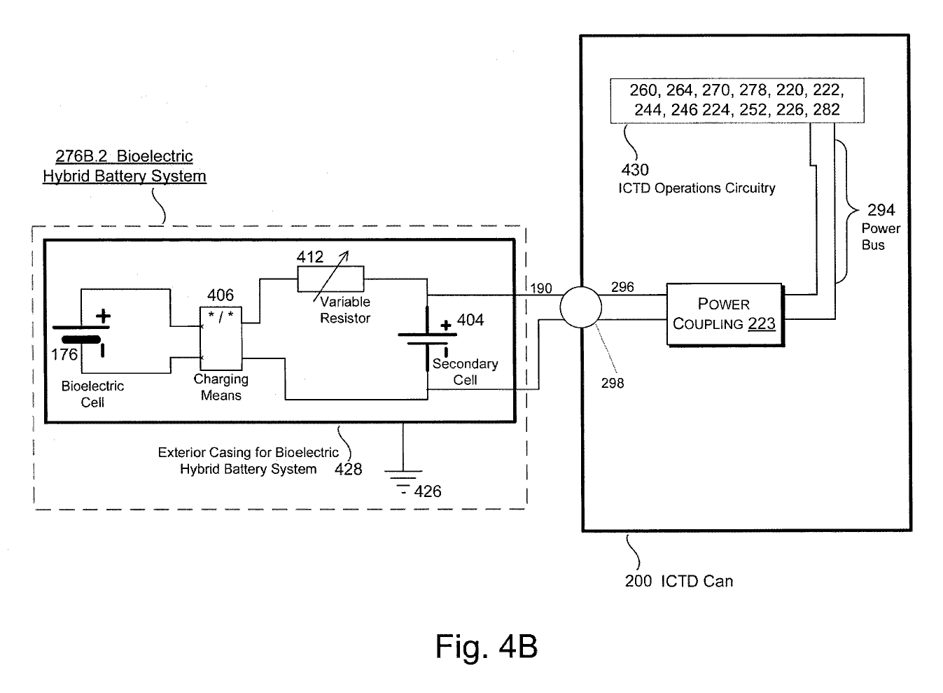

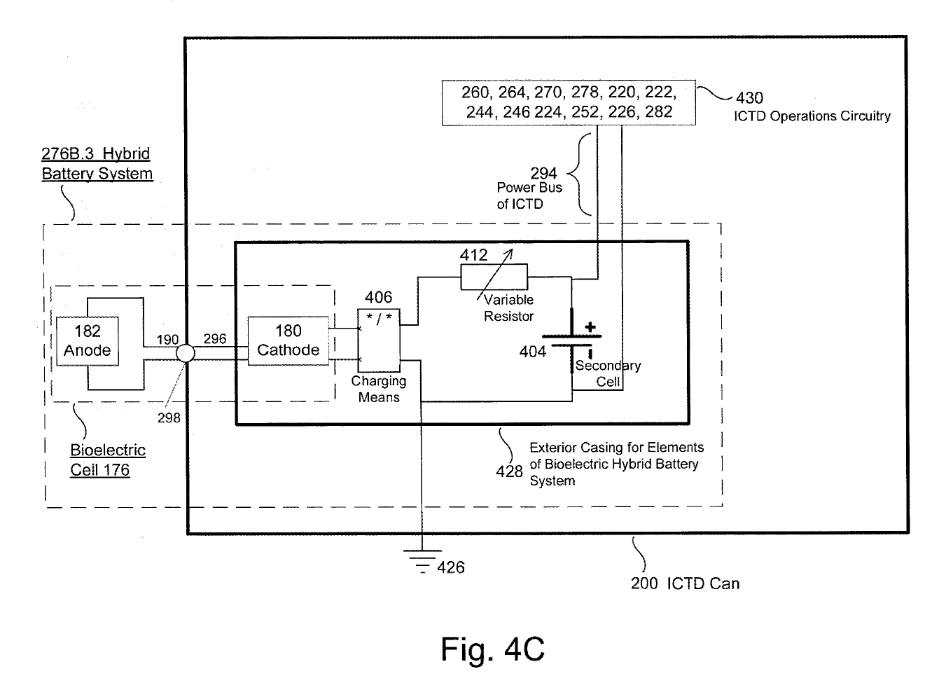

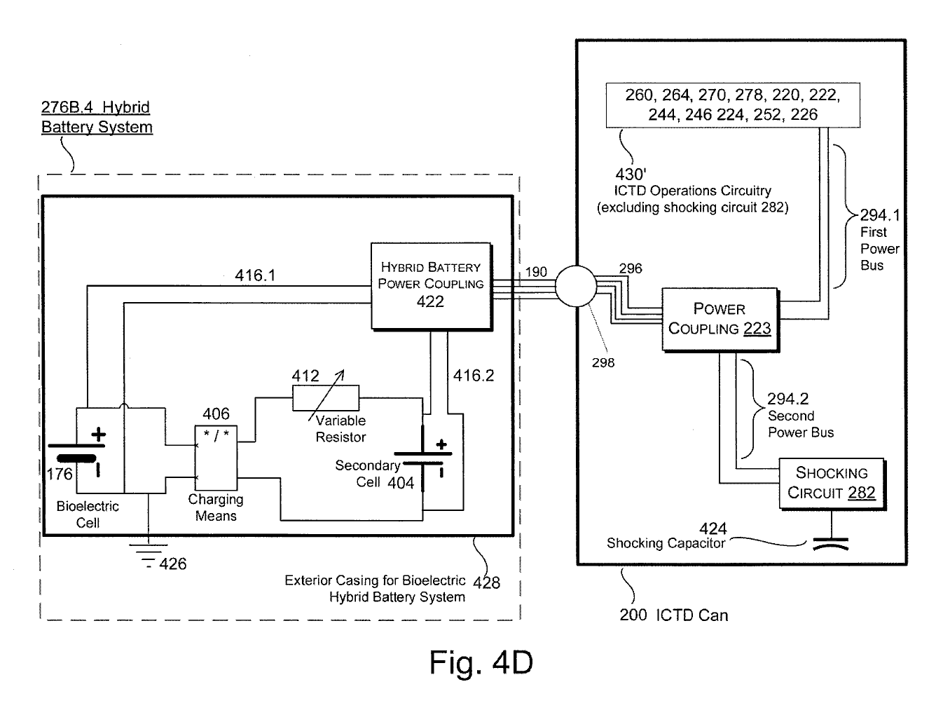

The present system and method for a hybrid battery system includes a bioelectric cell 176 implanted in the patient's body. In an embodiment, and as illustrated in FIG. 1, bioelectric cell 176 may be external to the case 200 (see FIG. 2) of ICTD 100 but is electrically coupled to ICTD 100. In particular, bioelectric cell 176 is coupled to a secondary, rechargeable cell 404 (see FIG. 4) which may be internal to ICTD 100. In an alternative embodiment, both bioelectric cell 176 and rechargeable cell 404 are external to ICTD 100, and together are coupled to ICTD 100.

In an alternative embodiment (not illustrated in FIG. 1), bioelectric cell 176 may be embedded or partially embedded within ICTD 100, provided both an anode material 182 and a cathode material 180 of bioelectric cell 176 are configured to receive a bodily fluid of the patient. For example, an anode 182 and cathode 180 (both discussed further below) of bioelectric cell 176 may be attached to, embedded within, project from, be contiguous with, or otherwise be part of external case 200 of ICTD 100.

A detailed discussion of exemplary bioelectric cells is presented in co-pending and commonly-owned U.S. patent application Ser. No. 11/737,307, entitled “Bioelectric Battery for Implantable Device Applications”, filed Apr. 19, 2007, which is incorporated by reference herein in its entirety. A partial discussion of some embodiments of a bioelectric cell 176 is included here to provide context and background, it being understood that many other embodiments are possible as well.

Bioelectric cells, also known as bioelectric batteries or biogalvanic cells, are implanted in the body and may rely on oxygen in internal body fluids for creating a voltage between an anode electrode 182 and a cathode electrode 180. Oxygen in the body fluids reacts with the anode 182 and consumes the anode 182, thereby creating an electric potential between the anode 182 and cathode 180 electrodes. Oxygen is present in the body in plentiful supply so the lifetime of the battery is limited only by the amount of anode material 182.

A first embodiment of a bioelectric cell will be described with reference to FIG. 1. A first embodiment of a bioelectric cell is generally shown at 176 in FIG. 1. Bioelectric cell 176 has a cathode electrode 180 and an anode electrode 182, which in an embodiment are built into a single unit. Cathode 180 and anode 182 are separated by an insulating member 184. Insulating member 184 may be a dielectric material including, for example and without limitation, silicone, polytetrafluoroethylene, or other dielectric polymer and may be formed in the shape of a cylindrical tube. Anode 182 may also be cylindrical in shape and inserted into a first end of insulating member 184. Cathode 180 may be in the form of a wire and may be coiled around insulating member 184.

Materials are chosen for anode 182 and cathode 180 that do not exhibit toxicity to the body of the organism in which they are implanted. Anode 182 is a reactive consumable metal that is consumed during the operation of the bioelectric cell and released into the body. Therefore it should be a material that is normally present in the body and of a size that when released into the body does not increase the levels of the material beyond a normally recommended level.

Anode material 182 should generate a high voltage when in reaction with oxygen. The material for anode 182 may include, but is not limited to, magnesium alloys. Magnesium alloys include magnesium along with aluminum, zinc, manganese, silver, copper, nickel, zirconium and/or rare earth elements, such as neodymium, gadolinium, and yttrium. Such magnesium alloys include, for example and without limitation, AZ61A supplied by Metal Mart International (5828 Smithway Street, Commerce, Calif. 90040) or AZ91E, EL21, or WE43 supplied by Magnesium Elektron (1001 College Street, Madison, Ill. 62060 USA).

The material for cathode 180 is a non-consumable metal including, for example and without limitation, platinum or titanium. Cathode 180 may be in the form of, for example and without limitation, a metal foil or wire. Cathode 180 may also have a coating that acts as a catalyst for the reaction at cathode 180. A coating increases the surface area of cathode 180, thereby resulting in a faster reaction and increased voltage generation. The coating may include, for example and without limitation, platinum black, iridium oxide (IrO2), ruthenium oxide (RuO2) or an IrO2/RuO2 mixture. For example, cathode 180 may be a platinum black coated platinum wire or an iridium oxide coated titanium wire. The coating may be applied using conventional methods including, without limitation, electrochemical deposition, thermal decomposition or sputtering.

The electrolyte for the bioelectric cell 176 may be a body fluid including, for example and without limitation, blood or other fluids extant in body cavities. When the electrolyte is a body fluid, the body fluid directly contacts cathode 180 and anode 182, such that oxygen dissolved in the body fluid is absorbed onto a surface of cathode 180 and reacts with anode 182.

A first end of a lead 190, such as a pacing lead with an IS-1 connection, extends from a second end of insulating member 184 and provides a current flow between anode 182 and cathode 180. Lead 190 further provides power to a load, including, for example and without limitation, an implantable medical device 100 or a secondary power cell 404 (not illustrated in FIG. 1, see FIG. 4), connected to a second end of lead 190. Exemplary implantable medical devices include, for example and without limitation, pacemakers, monitors or implantable cardioverter defibrillators (ICDs), and more generally any form of implantable cardiac therapy devices (ICTDs). Exemplary implantable medical devices further include implantable pumps and drug infusions devices.

Bioelectric cell 176 may be sufficient to power an implantable monitor; intrapericardial pacemaker, intraventricular pacemaker or standard pacemaker; or the background operations of ICTD 100. Bioelectric cell 176 may also be coupled to a rechargeable secondary cell 404 (not illustrated in FIG. 1, see FIG. 4) which may be internal to ICTD 100 or which may external to ICTD 100. When coupled to secondary cell 404, bioelectric cell 176 and secondary cell 404 together, possibly along with other associated electronics, may comprise a hybrid battery system. Such a hybrid battery system is discussed in more detail below.

In one embodiment of bioelectric cell 176, a magnesium alloy cylinder 182 is inserted into silicone tubing 184 and a platinum wire 180 is coiled around the silicone tubing. The magnesium alloy cylinder 182 and platinum wire 180 are connected to lead 190 to act as the anode electrode 182 and cathode electrode 180, respectively, of bioelectric cell 176. Magnesium from anode 182 and oxygen in the body fluids are slowly consumed as a current is generated. The platinum wire may be coated, such as with a platinum black coating. Alternatively, a titanium wire may be used as the cathode electrode 180. The titanium wire may be coated, such as with a platinum black, iridium oxide or ruthenium oxide coating.

The lifetime of anode electrode 182 may be five years, ten years, or in some embodiments even as long as twenty years. The exceptionally long lifetime of bioelectric cell 176 makes a bioelectric hybrid battery system 276B (not shown in FIG. 1, but discussed in conjunctions with FIGS. 2B, 2C, 4A-4D, and other figures below) an excellent choice for a power supply for ICTD 100. The long lifetime minimizes the need for surgical interventions to replace the ICTD power source.

Disclosed immediately above are exemplary embodiments of a bioelectric cell. Many other embodiments are possible consistent with the present system and method for a hybrid battery system which includes a bioelectric cell. Additional exemplary embodiments of a bioelectric cell are presented in above referenced U.S. patent application Ser. No. 11/737,307

Bioelectric cell 176 may be implanted anywhere in the body of an organism including, for example and without limitation, subcutaneously in the neck, the pectoral cavity, the superior vena cava, the intrapericardial space or the peritoneal cavity. Bioelectric cell 176 is implanted in tissue or blood vessels such that cathode 180 and anode 182 are in direct contact with body fluids. Therefore, the body fluids may act as the electrolyte for bioelectric cell 176.

In an alternative embodiment, bioelectric cell 176 may have an internal electrolyte (not illustrated in FIG. 1) in contact with anode 182 and cathode 180, where the internal electrolyte is not a bodily fluid of the patient. The internal electrolyte may be surrounded by a semipermeable membrane (not illustrated in FIG. 1) or other semipermeable material which permits diffusion or transfer of a replenishable organic material of the patient. For example, blood oxygen may diffuse from the patient's blood, across the membrane, through the internal electrolyte, and thereby reach anode 182 and cathode 180.

### 5. Functional Elements of an Exemplary ICTD

An implantable cardiac therapy device 100 may be referred to variously, and equivalently, throughout this document as an “implantable cardiac therapy device” (“ICTD”), an “implantable device”, a “stimulation device”, a “pacemaker”, a “monitor”, or an “implantable cardioverter defibrillator” (“ICD”), and the respective plurals thereof.

FIG. 2A shows an exemplary, simplified block diagram depicting various components of stimulation device 100. The stimulation device 100 can be capable of treating both fast and slow arrhythmias with stimulation therapy, including cardioversion, defibrillation, and pacing stimulation. The stimulation device can be solely or further capable of delivering stimuli to autonomic nerves. In addition to cardioversion, defibrillation, and pacing stimulation, stimulation device 100 is generally enabled to perform various supporting tasks, also referred to as “background tasks” or “background operations”. Background operations may include, for example and without limitation, sensing cardiac activity, sensing related physiological activity, analyzing cardiac activity or other physiological data, data storage and retrieval, and transmission of physiological data via radio frequency signals.

While a particular multi-chamber device is shown, it is to be appreciated and understood that this is done for illustration purposes only. For example, various methods may be implemented on a pacing device suited for single ventricular stimulation and not bi-ventricular stimulation. Thus, the techniques and methods described below can be implemented in connection with any suitably configured or configurable stimulation device. Accordingly, one of skill in the art could readily duplicate, eliminate, or disable the appropriate circuitry in any desired combination to provide a device capable of treating the appropriate chamber(s) or regions of a patient's heart with cardioversion, defibrillation, pacing stimulation, and/or autonomic nerve stimulation.

Housing 200 for stimulation device 100 is often referred to as the “can”, “case” or “case electrode”, and may be programmably selected to act as the return electrode for all “unipolar” modes. Housing 200 may further be used as a return electrode alone or in combination with one or more of the coil electrodes 126, 132 and 134 (see FIG. 1) for shocking purposes. Housing 200 further includes a connector (not shown) having a plurality of terminals 201, 202, 204, 206, 208, 212, 214, 216, 218, 221 (shown schematically and, for convenience, the names of the electrodes to which they are connected are shown next to the terminals).

To achieve right atrial sensing, pacing and/or autonomic stimulation, the connector includes at least a right atrial tip terminal (AR TIP) 202 adapted for connection to the atrial tip electrode 120. A right atrial ring terminal (AR RING) 201 is also shown, which is adapted for connection to the atrial ring electrode 121. To achieve left chamber sensing, pacing, shocking, and/or autonomic stimulation, the connector includes at least a left ventricular tip terminal (VL TIP) 204, a left atrial ring terminal (AL RING) 206, and a left atrial shocking terminal (AL COIL) 208, which are adapted for connection to the left ventricular tip electrode 122, the left atrial ring electrode 124, and the left atrial coil electrode 126, respectively. Connection to suitable autonomic nerve stimulation electrodes is also possible via these and/or other terminals (e.g., via a nerve stimulation terminal S ELEC 221).

To support right chamber sensing, pacing, shocking, and/or autonomic nerve stimulation, the connector further includes a right ventricular tip terminal (VR TIP) 212, a right ventricular ring terminal (VR RING) 214, a right ventricular shocking terminal (RV COIL) 216, and a superior vena cava shocking terminal (SVC COIL) 218, which are adapted for connection to the right ventricular tip electrode 128, right ventricular ring electrode 130, the RV coil electrode 132, and the SVC coil electrode 134, respectively. Connection to suitable autonomic nerve stimulation electrodes is also possible via these and/or other terminals (e.g., via the nerve stimulation terminal S ELEC 221).

At the core of the stimulation device 100 is a programmable microcontroller 220 that controls the various modes of stimulation therapy. As is well known in the art, microcontroller 220 typically includes a processor or microprocessor 231, or equivalent control circuitry, designed specifically for controlling the delivery of stimulation therapy, and may further include onboard memory 232 (which may be, for example and without limitation, RAM, ROM, PROM, one or more internal registers, etc.), logic and timing circuitry, state machine circuitry, and I/O circuitry.

Typically, microcontroller 220 includes the ability to process or monitor input signals (data or information) as controlled by a program code stored in a designated block of memory. The type of microcontroller is not critical to the described implementations. Rather, any suitable microcontroller 220 may be used that carries out the functions described herein. The use of microprocessor-based control circuits for performing timing and data analysis functions are well known in the art.

Representative types of control circuitry that may be used in connection with the described embodiments can include the microprocessor-based control system of U.S. Pat. No. 4,940,052 (Mann et al.), the state-machine of U.S. Pat. Nos. 4,712,555 (Thornander) and 4,944,298 (Sholder), all of which are incorporated by reference herein. For a more detailed description of the various timing intervals used within the stimulation device and their inter-relationship, see U.S. Pat. No. 4,788,980 (Mann et al.), also incorporated herein by reference.

FIG. 2A also shows an atrial pulse generator 222 and a ventricular pulse generator 224 that generate pacing stimulation pulses for delivery by the right atrial lead 104, the coronary sinus lead 106, and/or the right ventricular lead 108 via an electrode configuration switch 226. It is understood that in order to provide stimulation therapy in each of the four chambers of the heart (or to autonomic nerves or other tissue) the atrial and ventricular pulse generators, 222 and 224, may include dedicated, independent pulse generators, multiplexed pulse generators, or shared pulse generators. The pulse generators 222 and 224 are controlled by the microcontroller 220 via appropriate control signals 228 and 230, respectively, to trigger or inhibit the stimulation pulses.

Microcontroller 220 further includes timing control circuitry 233 to control the timing of the stimulation pulses (e.g., pacing rate, atrio-ventricular (e.g., AV) delay, atrial interconduction (AA) delay, or ventricular interconduction (VV) delay, etc.) as well as to keep track of the timing of refractory periods, blanking intervals, noise detection windows, evoked response windows, alert intervals, marker channel timing, etc., which is well known in the art.

Microcontroller 220 further includes an arrhythmia detector 234, a morphology detector 236, and optionally an orthostatic compensator and a minute ventilation (MV) response module (the latter two are not shown in FIG. 2A). These components can be utilized by the stimulation device 100 for determining desirable times to administer various therapies, including those to reduce the effects of orthostatic hypotension. The aforementioned components may be implemented in hardware as part of the microcontroller 220, or as software/firmware instructions programmed into the device and executed on the microcontroller 220 during certain modes of operation.

Microcontroller 220 further includes an AA delay, AV delay and/or W delay module 238 for performing a variety of tasks related to AA delay, AV delay and/or VV delay. This component can be utilized by the stimulation device 100 for determining desirable times to administer various therapies, including, but not limited to, ventricular stimulation therapy, bi-ventricular stimulation therapy, resynchronization therapy, atrial stimulation therapy, etc. The AA/AV/VV module 238 may be implemented in hardware as part of the microcontroller 220, or as software/firmware instructions programmed into the device and executed on the microcontroller 220 during certain modes of operation. Of course, such a module may be limited to one or more of the particular functions of AA delay, AV delay and/or W delay. Such a module may include other capabilities related to other functions that may be germane to the delays. Such a module may help make determinations as to fusion.

The microcontroller 220 of FIG. 2A also includes an activity module 239. This module may include control logic for one or more activity related features. For example, the module 239 may include an algorithm for determining patient activity level, calling for an activity test, calling for a change in one or more pacing parameters, etc. The module 239 may be implemented in hardware as part of the microcontroller 220, or as software/firmware instructions programmed into the device and executed on the microcontroller 220 during certain modes of operation. The module 239 may act cooperatively with the AA/AV/VV module 238.

Microcontroller 220 may also include a battery control module 286. Battery control module 286 may be used, for example, to control a battery 276 (which may be a hybrid battery 276H, and in particular a bioelectric hybrid battery 276B, illustrated in FIGS. 2B, 2C, and 4A-4D) as discussed in further detail below. Battery control 286 may be hardwired circuitry, or may be implemented as software or firmware running on microcontroller 220. Battery control 286 may be coupled to battery 276 via battery signal line 290 and battery control line 292. Battery signal line 290 may deliver to battery control 286 status or operational information regarding battery 276. Battery control line 292 may be used to change an operational state of battery 276. For example, battery control line 292 may deliver control signals from battery control 286 to battery 276. For example, in an embodiment where battery 276 is a hybrid battery, battery control 286 may send control signals to determine if a second cell is connected to a first cell for recharging of the second cell.

In an alternative embodiment, battery control 286 may be a separate module from microcontroller 220, but may be coupled to microcontroller 220. For example, separate module battery control 286 may obtain required ICTD operational status information from microcontroller 220. Or, for example, separate module battery control 286 may report battery status or battery operational information to microcontroller 220. In addition, separate module battery control 286 may also be coupled to battery 276.

In an alternative embodiment, battery control 286 may be implemented as an internal physical module of battery 276 (for example, battery control 286 may be implemented as a microchip which is situated internally to the exterior housing of battery 276). However, battery control 286 may still be coupled to microcontroller 220 via battery signal line 290 and battery control line 292. In an alternative embodiment, battery control functions of battery control 286 may be distributed across a first module which is part of battery 276, and one or more additional modules which are external to battery 276. The battery control module(s) external to battery 276 may for example be part of microcontroller 220.

Battery 276 is discussed in more detail below.

The electrode configuration switch 226 includes a plurality of switches for connecting the desired electrodes to the appropriate I/O circuits, thereby providing complete electrode programmability. Accordingly, switch 226, in response to a control signal 242 from the microcontroller 220, determines the polarity of the stimulation pulses (e.g., unipolar, bipolar, combipolar, etc.) by selectively closing the appropriate combination of switches (not shown) as is known in the art.

Atrial sensing circuits 244 and ventricular sensing circuits 246 may also be selectively coupled to the right atrial lead 104, coronary sinus lead 106, and the right ventricular lead 108, through the switch 226 for detecting the presence of cardiac activity in each of the four chambers of the heart. Accordingly, the atrial (ATR. SENSE) and ventricular (VTR. SENSE) sensing circuits, 244 and 246, may include dedicated sense amplifiers, multiplexed amplifiers, or shared amplifiers. Switch 226 determines the “sensing polarity” of the cardiac signal by selectively closing the appropriate switches, as is also known in the art. In this way, the clinician may program the sensing polarity independent of the stimulation polarity. The sensing circuits (e.g., 244 and 246) are optionally capable of obtaining information indicative of tissue capture.

Each sensing circuit 244 and 246 preferably employs one or more low power, precision amplifiers with programmable gain and/or automatic gain control, bandpass filtering, and a threshold detection circuit, as known in the art, to selectively sense the cardiac signal of interest. The automatic gain control enables the device 100 to deal effectively with the difficult problem of sensing the low amplitude signal characteristics of atrial or ventricular fibrillation.

The outputs of the atrial and ventricular sensing circuits 244 and 246 are connected to the microcontroller 220, which, in turn, is able to trigger or inhibit the atrial and ventricular pulse generators 222 and 224, respectively, in a demand fashion in response to the absence or presence of cardiac activity in the appropriate chambers of the heart. Furthermore, as described herein, the microcontroller 220 is also capable of analyzing information output from the sensing circuits 244 and 246 and/or the analog-to-digital (A/D) data acquisition system 252 to determine or detect whether and to what degree tissue capture has occurred and to program a pulse, or pulses, in response to such determinations. The sensing circuits 244 and 246, in turn, receive control signals over signal lines 248 and 250 from the microcontroller 220 for purposes of controlling the gain, threshold, polarization charge removal circuitry (not shown), and the timing of any blocking circuitry (not shown) coupled to the inputs of the sensing circuits 244, 246, as is known in the art.

For arrhythmia detection, the device 100 utilizes the atrial and ventricular sensing circuits, 244 and 246, to sense cardiac signals to determine whether a rhythm is physiologic or pathologic. In reference to arrhythmias, as used herein, “sensing” is reserved for the noting of an electrical signal or obtaining data (information), and “detection” is the processing (analysis) of these sensed signals and noting the presence of an arrhythmia. In some instances, detection or detecting includes sensing and in some instances sensing of a particular signal alone is sufficient for detection (e.g., presence/absence, etc.).

The timing intervals between sensed events (e.g., P-waves, R-waves, and depolarization signals associated with fibrillation which are sometimes referred to as “F-waves” or “Fib-waves”) are then classified by the arrhythmia detector 234 of the microcontroller 220 by comparing them to a predefined rate zone limit (i.e., bradycardia, normal, low rate VT, high rate VT, and fibrillation rate zones) and various other characteristics (e.g., sudden onset, stability, physiological sensors, and morphology, etc.) in order to determine the type of remedial therapy that is needed (e.g., bradycardia pacing, anti-tachycardia pacing, cardioversion shocks or defibrillation shocks, collectively referred to as “tiered therapy”).

Cardiac signals are also applied to inputs of an analog-to-digital (A/D) data acquisition system 252. The data acquisition system 252 is configured to acquire intracardiac electrogram (EGM) signals, convert the raw analog data into a digital signal, and store the digital signals for later processing and/or telemetric transmission to an external device 254. Data acquisition system 252 may be configured by microcontroller 220 via control signals 256. The data acquisition system 252 is coupled to the right atrial lead 104, the coronary sinus lead 106, the right ventricular lead 108 and/or the nerve stimulation lead 110 through the switch 226 to sample cardiac signals across any pair of desired electrodes.

The microcontroller 220 is further coupled to a memory 260 by a suitable data/address bus 262, wherein the programmable operating parameters used by the microcontroller 220 are stored and modified, as required, in order to customize the operation of the stimulation device 100 to suit the needs of a particular patient. Such operating parameters define, for example, pacing pulse amplitude, pulse duration, electrode polarity, rate, sensitivity, automatic features, arrhythmia detection criteria, and the amplitude, waveshape, number of pulses, and vector of each shocking pulse to be delivered to the patient's heart 102 within each respective tier of therapy. One feature may be the ability to sense and store a relatively large amount of data (e.g., from the data acquisition system 252), which data may then be used for subsequent analysis to guide the programming of the device.

Essentially, the operation of the ICTD control circuitry, including but not limited to pulse generators, timing control circuitry, delay modules, the activity module, battery utilization and related voltage and current control, and sensing and detection circuits, may be controlled, partly controlled, or fine-tuned by a variety of parameters, such as those indicated above which may be stored and modified, and may be set via an external ICTD programming device 254.

Advantageously, the operating parameters of the implantable device 100 may be non-invasively programmed into the memory 260 through a telemetry circuit 264 in telemetric communication via communication link 266 with the external device 254, which may be a general purpose computer, a dedicated ICTD programmer, a transtelephonic transceiver, or a diagnostic system analyzer. The microcontroller 220 activates the telemetry circuit 264 with a control signal 268. The telemetry circuit 264 advantageously allows intracardiac electrograms and status information relating to the operation of the device 100 (as contained in the microcontroller 220 or memory 260) to be sent to the external device 254 through an established communication link 266. The ICTD 100 may also receive human programmer instructions via the external device 254.

The stimulation device 100 can further include a physiological sensor 270, commonly referred to as a “rate-responsive” sensor because it is typically used to adjust pacing stimulation rate according to the exercise state of the patient. However, physiological sensor 270 may further be used to detect changes in cardiac output (see, e.g., U.S. Pat. No. 6,314,323, entitled “Heart stimulator determining cardiac output, by measuring the systolic pressure, for controlling the stimulation”, to Ekwall, issued Nov. 6, 2001, which discusses a pressure sensor adapted to sense pressure in a right ventricle and to generate an electrical pressure signal corresponding to the sensed pressure, an integrator supplied with the pressure signal which integrates the pressure signal between a start time and a stop time to produce an integration result that corresponds to cardiac output), changes in the physiological condition of the heart, or diurnal changes in activity (e.g., detecting sleep and wake states). Accordingly, the microcontroller 220 may respond by adjusting the various pacing parameters (such as rate, AA delay, AV delay, VV delay, etc.) at which the atrial and ventricular pulse generators, 222 and 224, generate stimulation pulses.

While shown as being included within the stimulation device 100, it is to be understood that the physiological sensor 270 may also be external to the stimulation device 100, yet still be implanted within or carried by the patient. Examples of physiological sensors that may be implemented in device 100 include known sensors that, for example, sense respiration rate, pH of blood, ventricular gradient, cardiac output, preload, afterload, contractility, hemodynamics, pressure, and so forth. Another sensor that may be used is one that detects activity variance, wherein an activity sensor is monitored diurnally to detect the low variance in the measurement corresponding to the sleep state. For a complete description of an example activity variance sensor, the reader is directed to U.S. Pat. No. 5,476,483 (Bornzin et al.), issued Dec. 19, 1995, which patent is hereby incorporated by reference.

More specifically, physiological sensors 270 optionally include sensors for detecting movement and minute ventilation in the patient. Physiological sensors 270 may include a position sensor and/or a minute ventilation (MV) sensor to sense minute ventilation, which is defined as the total volume of air that moves in and out of a patient's lungs in a minute. Signals generated by the position sensor and MV sensor are passed to the microcontroller 220 for analysis in determining whether to adjust the pacing rate, etc. The microcontroller 220 monitors the signals for indications of the patient's position and activity status, such as whether the patient is climbing upstairs or descending downstairs or whether the patient is sitting up after lying down.

Stimulation device 100 additionally includes battery 276 that provides operating power to all of the circuits shown in FIG. 2A, as well as to any additional circuits which may be present in alternative embodiments. Operating power in the form of electrical current and/or voltage may be provided via a power bus or power buses 294, depicted in FIG. 2A as a first power bus 294.1 and a second power bus 294.2. In FIG. 2A, the connection(s) of power bus(es) 294 to other elements of ICTD 100 for purposes of powering those elements is not illustrated, but is implied by the dotted end-lines of bus(es) 294.

For stimulation device 100, which employs shocking therapy, the battery 276 is capable of operating at low current drains for long periods of time (e.g., preferably less than 10 μAmps), and is capable of providing high-current pulses (for capacitor charging) when the patient requires a shock pulse (e.g., preferably, in excess of 2 Amps, at voltages above 2 volts, for periods of 10 seconds or more). In an embodiment, discussed in detail below, battery 276 may be configured to provide a current as high as 3 to 4.5 Amps and/or unloaded voltages in excess of 4 volts, for rapid charging of shocking circuitry. Battery 276 also desirably has a predictable discharge characteristic so that elective replacement time can be determined.

In an embodiment, battery 276 may be a hybrid battery system comprised of dual types of cells, as described further below. Such a hybrid battery system may provide power via a plurality of power buses, such as buses 249.1 and 294.2 of FIG. 2A. In an embodiment, each power bus may be configured to deliver different voltages, different currents, and/or different power levels. Battery 276 may be monitored and/or controlled via battery control 286, as discussed in part above, and as also discussed further below.

Further embodiments of a hybrid battery 276 employing a bioelectric cell 176 are discussed below.

The stimulation device 100 can further include magnet detection circuitry (not shown), coupled to the microcontroller 220, to detect when a magnet is placed over the stimulation device 100. A magnet may be used by a clinician to perform various test functions of the stimulation device 100 and/or to signal the microcontroller 220 that the external programmer 254 is in place to receive or transmit data to the microcontroller 220 through the telemetry circuit 264.

The stimulation device 100 further includes an impedance measuring circuit 278 that is enabled by the microcontroller 220 via a control signal 280. The known uses for an impedance measuring circuit 278 include, but are not limited to, lead impedance surveillance during the acute and chronic phases for proper lead positioning or dislodgement; detecting operable electrodes and automatically switching to an operable pair if dislodgement occurs; measuring respiration or minute ventilation; measuring thoracic impedance for determining shock thresholds; detecting when the device has been implanted; measuring stroke volume; and detecting the opening of heart valves, etc. The impedance measuring circuit 278 is advantageously coupled to the switch 226 via circuit line(s) 291 so that any desired electrode may be used.

In the case where the stimulation device 100 is intended to operate as an implantable cardioverter/defibrillator (ICTD) device, it detects the occurrence of an arrhythmia, and automatically applies an appropriate therapy to the heart aimed at terminating the detected arrhythmia. To this end, the microcontroller 220 further controls a shocking circuit 282 by way of a control signal 284. The shocking circuit 282 generates shocking pulses of low (e.g., up to approximately 0.5 J), moderate (e.g., approximately 0.5 J to approximately 10 J), or high energy (e.g., approximately 11 J to approximately 40 J), as controlled by the microcontroller 220. Such shocking pulses are applied to the patient's heart 102 through at least two shocking electrodes, and as shown in this embodiment, selected from the left atrial coil electrode 126, the RV coil electrode 132, and/or the SVC coil electrode 134. As noted above, the housing 200 may act as an active electrode in combination with the RV coil electrode 132, or as part of a split electrical vector using the SVC coil electrode 134 or the left atrial coil electrode 126 (i.e., using the RV electrode as a common electrode). Other exemplary devices may include one or more other coil electrodes or suitable shock electrodes (e.g., a LV coil, etc.). Shocking circuit 282 either has within it, or is coupled to, one or more shocking capacitors. The shocking capacitor(s) may be used to store up energy, and then release that energy, during the generation of shocking pulses.

Cardioversion level shocks are generally considered to be of low to moderate energy level (where possible, so as to minimize pain felt by the patient), and/or synchronized with an R-wave and/or pertaining to the treatment of tachycardia. Defibrillation shocks are generally of moderate to high energy level (i.e., corresponding to thresholds in the range of approximately 5 J to approximately 40 J), delivered asynchronously (since R-waves may be too disorganized), and pertaining exclusively to the treatment of fibrillation. Accordingly, microcontroller 220 is capable of controlling the synchronous or asynchronous delivery of the shocking pulses.

### 6. ICTD Programmer

As indicated above, the operating parameters of the implantable device 100 may be non-invasively programmed into the memory 260 through a telemetry circuit 264 in telemetric communication via communication link 266 with the external device 254. The external device 254 may be a general purpose computer running custom software for programming the ICTD 100, a dedicated external programmer device of ICTD 100, a transtelephonic transceiver, or a diagnostic system analyzer. Generically, all such devices may be understood as embodying computers, computational devices, or computational systems with supporting hardware or software which enable interaction with, data reception from, and programming of ICTD 100.

Throughout this document, where a person is intended to program or monitor ICTD 100 (where such person is typically a physician or other medical professional or clinician), the person is always referred to as a “human programmer” or as a “user”. The term “human programmer” may be viewed as synonymous with “a person who is a user of an ICTD programming device”, or simply with a “user”. Any other reference to “programmer” or similar terms, such as “ICTD programmer”, “external programmer”, “programming device”, etc., refers specifically to the hardware, firmware, software, and/or physical communications links used to interface with and program ICTD 100.

The terms “computer program”, “computer code”, and “computer control logic” are generally used synonymously and interchangeably in this document to refer to the instructions or code which control the behavior of a computational system. The term “software” may be employed as well, it being understood however that the associated code may in some embodiments be implemented via firmware or hardware, rather than as software in the strict sense of the term (e.g., as computer code stored on a removable medium, or transferred via a network connection, etc.).

A “computer program product” or “computational system program product” is a medium (for example, a magnetic disk drive, magnetic tape, optical disk (e.g., CD, DVD), firmware, ROM, PROM, flash memory, a network connection to a server from which software may be downloaded, etc) which is suitable for use in a computer or computation system, or suitable for input into a computer or computational system, where the medium has control logic stored therein for causing a processor of the computational system to execute computer code or a computer program. Such medium, also referred to as “computer program medium”, “computer usable medium”, and “computational system usable medium”, are discussed further below.

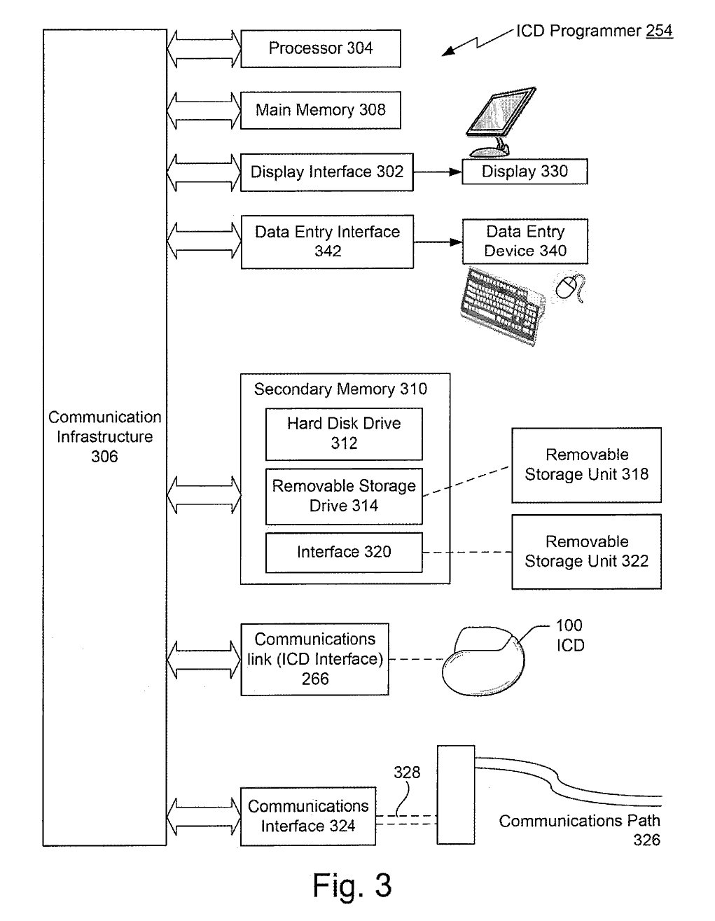

FIG. 3 presents a system diagram representing an exemplary computer, computational system, or other programming device, which will be referred to for convenience as ICTD programmer 254. It will be understood that while the device is referred to an “ICTD programmer”, indicating that the device may send programming data, programming instructions, programming code, and/or programming parameters to ICTD 100, the ICTD programmer 254 may receive data from ICTD 100 as well, and may display the received data in a variety of formats, analyze the received data, store the received data in a variety of formats, transmit the received data to other computer systems or technologies, and perform other tasks related to operational and/or physiologic data received from ICTD 100.

ICTD programmer 254 includes one or more processors, such as processor 304. Processor 304 is used for standard computational tasks well known in the art, such as retrieving instructions from a memory, processing the instructions, receiving data from memory, performing calculations and analyses on the data in accordance with the previously indicated instructions, storing the results of calculations back to memory, programming other internal devices within ICTD programmer 254, and transmitting data to and receiving data from various external devices such as ICTD 100.

Processor 304 is connected to a communication infrastructure 306 which is typically an internal communications bus of ICTD programmer 254; however, if ICTD programmer 254 is implemented in whole or in part as a distributed system, communication infrastructure 306 may further include or may be a network connection.

ICTD programmer 254 may include a display interface 302 that forwards graphics, text, and other data from the communication infrastructure 306 (or from a frame buffer not shown) for display on a display unit 330. The display unit may be, for example, a CRT, an LCD, or some other display device. Display unit 330 may also be more generally understood as any device which may convey data to a human programmer.

Display unit 330 may also be used to present a user interface which displays internal features of, operating modes or parameters of, or data from ICTD 100. The user interface presented via display unit 330 of ICTD programmer 254 may include various options that may be selected, deselected, or otherwise changed or modified by a human programmer of ICTD 100. The options for programming the ICTD 100 may be presented to the human programmer via the user interface in the form of buttons, check boxes, menu options, dialog boxes, text entry fields, or other icons or means of visual display well known in the art.

ICTD programmer 254 may include a data entry interface 342 that accepts data entry from a human programmer via data entry devices 340. Such data entry devices 340 may include, for example and without limitation, a keyboard, a mouse, a touchpad, a touch-sensitive screen, a microphone for voice input, or other means of data entry, which the human programmer uses in conjunction with display unit 330 in a manner well known in the art. For example, either a mouse or keystrokes entered on a keyboard may be used to select check boxes, option buttons, menu items, or other display elements indicating human programmer choices for programming ICTD 100. Direct text entry may be employed as well. Data entry device 340 may also take other forms, such as a dedicated control panel with specialized buttons and/or other mechanical elements or tactile sensitive elements for programming ICTD 100.

In the context of the present system and method, display interface 302 may present on display unit 330 a variety of data related to patient cardiac function and performance, and also data related to the present operating mode, operational state, or operating parameters of ICTD 100. Modifications to ICTD 100 operational state(s) may be accepted via data entry interface 342 and data entry device 340. In general, any interface means which enables a human programmer to interact with and program ICTD 100 may be employed. In one embodiment, for example, a visual data display may be combined with tactile data entry via a touch-screen display.

In another embodiment, a system of auditory output (such as a speaker or headset and suitable output port for same, not shown) may be employed to output data relayed from ICTD 100, and a system of verbal input (such as a microphone and suitable microphone port, not shown) may be employed to program ICTD 100. Other modes of input and output means may be employed as well including, for example and without limitation, a remote interaction with ICTD 100, viewing printed data which has been downloaded from ICTD 100, or the programming of ICTD 100 via a previously coded program script.

All such means of receiving data from ICTD 100 and/or programming ICTD 100 constitute an interface 302, 330, 342, 340 between ICTD 100 and a human programmer of ICTD 100, where the interface is enabled via both the input/output hardware (e.g., display screen, mouse, keyboard, touchscreen, speakers, microphone, input/output ports, etc.) and the hardware, firmware, and/or software of ICTD programmer 254.

ICTD programmer 254 also includes a main memory 308, preferably random access memory (RAM), and may also include a secondary memory 310. The secondary memory 310 may include, for example, a hard disk drive 312 and/or a removable storage drive 314, representing a floppy disk drive, a magnetic tape drive, an optical disk drive, etc. The removable storage drive 314 reads from and/or writes to a removable storage unit 318 in a well known manner. Removable storage unit 318 represents a magnetic disk, magnetic tape, optical disk, etc. which is read by and written to by removable storage drive 314. As will be appreciated, the removable storage unit 318 includes a computer usable storage medium having stored therein computer software and/or data.

In alternative embodiments, secondary memory 310 may include other similar devices for allowing computer programs or other instructions to be loaded into ICTD programmer 254. Such devices may include, for example, a removable storage unit 322 and an interface 320. Examples of such may include a program cartridge and cartridge interface (such as that found in video game devices), a removable memory chip (such as an erasable programmable read only memory (EPROM), programmable read only memory (PROM), or flash memory) and associated socket, and other removable storage units 322 and interfaces 320, which allow software and data to be transferred from the removable storage unit 322 to ICTD programmer 254.

ICTD programmer 254 also contains a communications link 266 to ICTD 100, which may be comprised in part of a dedicated port of ICTD programmer 254. From the perspective of ICTD programmer 254, communications link 266 may also be viewed as an ICTD interface. Communications link 266 enables two-way communications of data between ICTD programmer 254 and ICTD 100. Communications link 266 has been discussed above (see the discussion of FIG. 2A).

ICTD programmer 254 may also include a communications interface 324. Communications interface 324 allows software and data to be transferred between ICTD programmer 254 and other external devices (apart from ICTD 100). Examples of communications interface 324 may include a modem, a network interface (such as an Ethernet card), a communications port, a Personal Computer Memory Card International Association (PCMCIA) slot and card, a USB port, an IEEE 1394 (FireWire) port, etc. Software and data transferred via communications interface 324 are in the form of signals 328 which may be electronic, electromagnetic, optical (e.g., infrared) or other signals capable of being received by communications interface 324. These signals 328 are provided to communications interface 324 via a communications path (e.g., channel) 326. This channel 326 carries signals 328 and may be implemented using wire or cable, fiber optics, a telephone line, a cellular link, an radio frequency (RF) link, in infrared link, and other communications channels.

The terms “computer program medium”, “computer usable medium”, and “computational system usable medium” are used, synonymously, to generally refer to media such as removable storage drive 314 and removable storage unit 381, a hard disk installed in hard disk drive 312, a secondary memory interface (such as a flash memory port, USB port, FireWire port, etc.) and removable storage unit 322 (such as flash memory), and removable storage units 318 and 322. These computer program products or computational system program products provide software to ICTD programmer 254.

It should be noted, however, that it is not necessarily the case that the necessary software, computer code, or computer program (any of which may also referred to as computer control logic) be loaded into ICTD programmer 254 via a removable storage medium. Such computer program may be loaded into ICTD programmer 254 via communications link 328, or may be stored in memory 308 of ICTD programmer 254. Computer programs are stored in main memory 308 and/or secondary memory 310. Computer programs may also be received via communications interface 324.

Accordingly, such computer programs represent controllers of ICTD programmer 254, and thereby controllers of ICTD 100. Software may be stored in a computer program product and loaded into ICTD programmer 254 using removable storage drive 314, hard drive 312, secondary memory interface 320, or communications interface 324.

In an embodiment of the present system and method, ICTD programmer 254 may be used to modify ICTD operating parameters of battery control 286. In this way, ICTD programmer 254 may be used to modify the operations of a battery 276, such as a bioelectric hybrid battery 276B discussed in further detail below.

### 7. Hybrid Battery with Bioelectric Cell

A hybrid battery system which includes a bioelectric cell may be referred to synonymously as a “bioelectric hybrid battery system”, or simply as a “bioelectric hybrid battery”.

In an embodiment illustrated in FIGS. 2B and 2C, battery 276 of ICTD 100 may be an exemplary bioelectric hybrid battery system 276B, also referred to simply as bioelectric hybrid battery 276B. Bioelectric hybrid battery 276B may be comprised of a bioelectric cell 176 (already described above in conjunction with FIG. 1) and a secondary cell 404 (discussed in further detail in conjunction with FIGS. 4A-4D below).

As discussed in conjunction with FIG. 1 above, bioelectric cell 176 of bioelectric hybrid battery 276B may be external to can 200 of ICTD 100. In an embodiment, secondary cell 404 may be located internally to can 200 of ICTD 100.

Bioelectric cell 176 may be electrically coupled to ICTD 100 partly via lead 190, such as a pacing lead with an IS-1 connection. Lead 190 may connect to battery terminal 298 of ICTD 100. In turn, an electrical connection between bioelectric cell 176 and secondary cell 404 may be completed by internal power line 296 of ICTD 100. Internal power line 296 couples battery terminal 298 to secondary cell 404.

In the embodiment illustrated in FIG. 2B, internal buses 294.1 and 294.2 functional in a manner substantially the same or similar to that already discussed above in conjunction with FIG. 2A. If a single bus 294 is employed, bus 294 may deliver a voltage from secondary cell 404 of bioelectric hybrid battery 276B. If two or more buses are employed, such as buses 294.1 and 294.2, then each bus may deliver a voltage from a different source and at a different level. For example, first bus 294.1 may deliver voltage and current which is delivered from bioelectric cell 176, while second bus 294.2 may deliver voltage and current which is delivered from secondary cell 404.

Similarly, battery signal line 290 and battery control line 292 may function in a manner substantially the same or similar to that already discussed above, providing suitable monitoring and control connection(s) between bioelectric hybrid battery 276B and battery control 286.

Bioelectric hybrid battery system 276B may include other elements and components in addition to bioelectric cell 176, secondary cell 404, and associated power lines, control lines, and signaling lines. Exemplary additional elements are discussed further below in conjunction with FIGS. 4A-4D.

An alternative exemplary embodiment of bioelectric hybrid battery system 276B is illustrated in FIG. 2C. In this exemplary embodiment, both bioelectric cell 176 and secondary cell 404 are external to ICTD 100, and are housed within a shared external casing 428 (discussed in more detail in conjunction with FIGS. 4A-4D, below). Bioelectric cell 176 and secondary cell 404 are illustrated in FIG. 2C as being electrically coupled via a simple coupling 299, however, this is representational only. Typically, additional elements may be required to couple bioelectric cell 176 and secondary cell 404, including for example and without limitation a voltage converter such as a DC-to-DC converter. These elements are discussed in more detail below in conjunction with FIGS. 4A-4D.

Bioelectric hybrid battery system 276B may be coupled to ICTD 100 partly via lead 190. Additional leads may be used as well (not illustrated in FIG. 2C), possibly along with signaling and control lines (also not illustrated in FIG. 2C). Lead 190 may connect to battery terminal 298 of ICTD 100. In turn, an electrical coupling may be completed by internal power line 296 between bioelectric hybrid battery 276B and an internal power coupling 223 of ICTD 100. Internal power coupling 223 may be used to route electrical power supplied by external bioelectric hybrid battery 276B to various elements within ICTD 100. Power coupling 223 may be, for example, a digitally controlled switch that receives inputs from cell 176 and cell 404 and connects a selected cell to provide power to selected elements of ICTD 100 including operations circuitry and/or shocking circuitry.

In the exemplary embodiment illustrated in FIG. 2C, internal buses 294.1 and 294.2 function in a manner substantially the same or similar to that already discussed above in conjunction with FIG. 2A, routing power, including possibly power at different voltages or different currents, from power coupling 223 to elements of ICTD 100.

Similarly, battery signal line 290 and battery control line 292 may function in a manner substantially the same or similar to that already discussed above. That is, battery signal line 290 and battery control line 292 may provide suitable monitoring and control connection(s) between bioelectric hybrid battery 276B and battery control 286. In an embodiment, monitoring and control connections may be routed via power coupling 223. In an alternative embodiment, other monitoring and control connections may be used to route monitoring and control signals to and from bioelectric hybrid battery 276B, without routing through power coupling 223.

In an alternative embodiment of the present system and method, bioelectric hybrid battery system 276B may be substantially contained within ICTD 100. In such an embodiment, secondary cell 404 will typically be contained substantially or completely within case 200 of ICTD 100. Similarly, coupling elements 299 (discussed in further detail below in conjunction with FIGS. 4A-4D) will typically be contained substantially or completely within case 200 of ICTD 100.

In such an embodiment (that is, an embodiment where bioelectric hybrid battery 276B is substantially contained within ICTD 100), several elements or all elements of bioelectric cell 176 may be completely or substantially contained within case 200 or ICTD 100. However, bioelectric cell 176 is configured so that a replenishable bodily substance of the patient can reach anode 182 and cathode 180 of bioelectric cell 176. Typically, this may be achieved by configuring anode 182 and cathode 180 to receive a bodily fluid of the patient, such as the patient's blood.

In an embodiment, anode 182 and/or cathode 180 of bioelectric cell 176 may be attached to, embedded within, project from, be contiguous with, or otherwise be part of external case 200 of ICTD 100, thereby allowing access to bodily fluids which may surround case 200. In an alternative embodiment, anode 182 and/or cathode 180 may be configured to be interior to external case 200 of ICTD 100. Channels, pipes, or other fluid conveying elements may run through ICTD 100, and permit bodily fluids to reach anode 182 and/or cathode 180 of bioelectric cell 176.

### 8. Further Elements of Hybrid Battery with Bioelectric Cell

FIGS. 4A-4D present schematic diagrams of exemplary bioelectric hybrid battery systems 276B according to the present system and method. FIGS. 4A-4D also includes some elements of exemplary connections between exemplary hybrid battery systems 276B and other elements of ICTD 100.

FIG. 4A is a block diagram of an exemplary bioelectric hybrid battery system 276B.1 according to an embodiment of the present system and method. Bioelectric hybrid battery system 276B.1 may be comprised of an bioelectric cell 176 and a secondary cell 404. In an alternative embodiment, two or more bioelectric cells 176 may be employed in place of just a single bioelectric cell 176. In an alternative embodiment, two or more secondary cells 404 may be employed in place of just a single secondary cell 404.

Exemplary embodiments of bioelectric cell 176 have already been described above. Other embodiments are possible as well. In an embodiment, secondary cell 404 may be a lithium ion polymer cell. The present system and method may enjoy several advantages due to the specific selection secondary cells. These advantages are discussed in detail below in the section entitled “Choice of Secondary Power Cell”.

In an embodiment, and as illustrated in FIG. 4A, bioelectric cell 176 may be external to the can 200 of ICTD 100, while secondary cell 404 may be internal to the can 200 of ICTD 100. Bioelectric cell 176 may be coupled to ICTD 100 via lead 190, which may connected to battery terminal 298. Bioelectric cell 176 may be further coupled to secondary cell 404 via ICTD internal power line 296, which may also be coupled to battery terminal 298.

Bioelectric cell 176 and secondary cell 404 may be further coupled by charging means 406. Further coupled between charging means 406 and secondary cell 404 may be an variable resistor 412. In an embodiment, and as shown in FIG. 4A, bioelectric cell 176 and secondary cell 404 may be coupled in parallel. Hybrid bioelectric battery system 276B.1 may include an internal power bus 420 configured to deliver power from hybrid bioelectric battery system 276B.1 to elements of ICTD 100.

In FIG. 4A, the dashed box contains those elements which may comprise exemplary bioelectric hybrid battery system 276B.1. Bioelectric hybrid battery system 276B.1 is comprised of bioelectric cell 176, secondary cell 404, and charging means 406. Bioelectric hybrid battery system 276B.1 may therefore be comprised of elements which are both internal to and external to ICTD 100. Bioelectric hybrid battery system 176B may be further comprised of other elements including, for example and without limitation, variable resistor 412, internal power bus 420, and a case 428. Case 428 may enclose some elements of bioelectric hybrid battery system 276B.1, as illustrated with exemplary embodiments throughout this document.

Bioelectric hybrid battery system 276B.1 may be coupled to an ICTD power bus 294. In turn, power bus 294 may be connected to numerous elements of ICTD 100 already discussed above. These elements may include, for example and without limitation, memory 260, telemetry circuit 264, physiological sensor 270, impedance measuring circuit 278, microcontroller 220, atrial pulse generator 222, atrial sensing circuits 244, ventricular sensing circuits 246, analog-to-digital converter 252, electrode configuration switch 226, and shocking circuit 282. Collectively, these elements and similar elements of ICTD 100 may be referred to as ICTD operations circuitry 430. ICTD operations circuitry 430 is thereby powered by bioelectric hybrid battery system 276B.1.

Additional elements of hybrid battery system 276B.1 may include connections or leads to grounding elements 426. Grounding elements 426 may, for example, be the exterior housing or “can” 200 of ICTD 100.

Variable resistor 412 may also be coupled between charging means 416 and secondary cell 404. As discussed further below, secondary cell 404 may be charged from bioelectric cell 176 via an unregulated charging process, meaning that secondary cell 404 can received current at a steady rate without risk of damage to secondary cell 404, and without risk of harm to the patient in whom ICTD 100 is implanted. However, bioelectric cell(s) 176 may only be able to discharge current provided the current flow from bioelectric cell(s) 176 is below a certain rate, for example, typically on the order of 100 μAmps. Variable resistor 412 may therefore serve the purpose of limiting a rate at which current is drawn from bioelectric cell 176. The exact resistance of variable resistor 412 may be slowly varied over an extended period of time (such as over periods of several months) via control circuitry.

As used herein, an “unregulated charging process” is a charging process where there is no requirement for circuitry or for a method to monitor and adjust the charging process on account of the possibility of overcharge of secondary cell 404. The term “unregulated charging process” is not intended to refer to the operation of charging means 406 (discussed further below), but only to the regulation of the charging of secondary cell 404 to prevent an overcharging condition. For example, a person skilled in the art will recognize that charging means 406 may be implemented as a regulated DC-to-DC converter which will use feedback to regulate the output voltage at a desired voltage level. Such regulation is separate and apart from overcharge regulation.

Further, even with an unregulated charging process, it may be desirable to control the rate of current flow, for example, to set a maximum limit to the current drawn from bioelectric cell 176 to secondary cell 404. This limit may be set, for example, via variable resistor 412. The phrase “unregulated charging process” may be further understood to mean that such a maximum limit to the current flow, once set, does not need to be further regulated or controlled over time in order to prevent overcharge or damage to secondary cell 404. The maximum permitted current flow from bioelectric cell 176 to secondary cell 404 may be set, for example, as part of a fixed design element of hybrid battery system 276.H, or may be set on a per unit basis during an initial configuration or set up of hybrid battery system 276.H.

As discussed further below, the ability to charge secondary cell 404 via an unregulated charging process may be enabled by a choice of a specific type of secondary cell 404, such as for example a lithium ion polymer cell.

Bioelectric cell 176 may also be coupled via charging means 406 to secondary cell 404. Bioelectric cell 176 and secondary cell 404 are configured so that secondary cell 404 may be continuously charged via charging means 406.

It is an advantage of the present system and method that because secondary cell 404 may be a lithium ion polymer cell, it is possible to continually charge secondary cell 404. Other possible types of secondary cells, such as, for example, a standard lithium ion cell (which is not a lithium ion polymer cell), may require careful regulation of the charging process. For example, regulation may be required to ensure that the other types of secondary cells do not charge too rapidly. Charging too rapidly may damage these other types of secondary cells and may even result in rupture or burning of the secondary cell.

However, when a secondary cell 404 is a lithium ion polymer cell, it is possible to charge secondary cell 404 from bioelectric cell 176 according to an unregulated charging process, as that term is defined above. Put another way, bioelectric cell 176 may transfer power to secondary cell 404 as rapidly as secondary cell 404 is capable of absorbing the power. As a result, there is no requirement for complex regulation circuitry to regulate, control, or limit the charging process. Secondary cell 404 may be continuously charged from bioelectric cell 176, or put another way, secondary cell 404 may be charged from bioelectric cell 176 via an unregulated charging process.

Charging means 406 may be, for example, a DC-to-DC converter. In an embodiment, no other charging circuitry is required to charge secondary cell 404 from bioelectric cell 176. In an alternative embodiment, variable resistor 412 may limit the rate of current flow to secondary cell 404.

In an embodiment of the present system and method, bioelectric cell 176 may put out a voltage anywhere in a range of approximately 0.5 volts up to 2 volts, depending on the exact configuration of bioelectric cell 176. DC-to-DC converter 406 steps up this voltage to a voltage above four volts. In this way secondary cell 404 is maintained at a voltage, such as, for example, approximately 4.1 to 4.2 volts, which is substantially above the voltage of bioelectric cell 176. The output voltage of charging means 406 may therefore be set at approximately 4.1 to 4.2 volts, so that secondary cell 404 can be maintained at a voltage level higher than 4.0 volts. In an embodiment of the present system and method, DC-to-DC converter 406 is configured for high-efficiency voltage conversion, resulting in minimal energy loss.

FIG. 4B presents a block diagram of another exemplary bioelectric hybrid battery system 276B.2 according to an embodiment of the present system and method. Many elements of exemplary bioelectric hybrid battery system 276B.2 are substantially the same or similar to those presented in conjunction with exemplary bioelectric hybrid battery system 276B.1 discussed above (see FIG. 4A), and a detailed discussion of those elements will not be repeated here.

In FIG. 4B, the dashed box (labeled 276B.2) contains those elements which may comprise exemplary bioelectric hybrid battery system 276B.2. Exemplary bioelectric hybrid battery system 276B.2 is entirely external to case 200 of ICTD 100. Therefore, in an embodiment, all elements of bioelectric hybrid battery system 276B.2 may be packaged together, for example, either within or on the surface of exterior case 428. These elements may include bioelectric cell 176, charging means 406, secondary cell 404, variable resistor 412, and various internal connectors and leads, and possibly other electrical and control elements (not illustrated in FIG. 4B). In an alternative embodiment, some elements may be on a surface of or external to exterior case 428. For example, either or both of cathode 180 and/or anode 182 of bioelectric cell 176 may be on a surface of case 428. Or, for example, either or both of cathode 180 and/or anode 182 of bioelectric cell 176 may be external to case 428, and coupled to bioelectric hybrid battery system 276B.2 via an electrical lead (not shown).

Exemplary bioelectric hybrid battery system 276B.2 may be coupled to ICTD 100 via lead 190, thereby providing electrical power to ICTD 100. Lead 190 may connect to battery terminal 298 of ICTD 100. From there, internal power line 296 delivers power to power coupling 223. Power coupling 223 may deliver power via power bus 294 to ICTD operations circuitry 430.

FIG. 4C presents a block diagram of another exemplary bioelectric hybrid battery system 276B.3 according to an embodiment of the present system and method. Many elements of exemplary bioelectric hybrid battery system 276B.3 are substantially the same or similar to those presented in conjunction with exemplary bioelectric hybrid battery systems 276B.1, 276B.2 discussed above (see FIGS. 4A and 4B), and a detailed discussion of those elements will not be repeated here.

In FIG. 4C, the outer dashed box (labeled 276B.3) contains those elements which may comprise exemplary bioelectric hybrid battery system 276B.3. The inner dashed box (labeled 176) contains those elements which may comprise bioelectric cell 176 of bioelectric hybrid battery system 276B.3.

Anode 182 of bioelectric hybrid battery system 276B.3 is external to case 428 which contains some elements of bioelectric hybrid battery system 276B.3. Anode 182 is further external to case 200 of ICTD 100, and in an embodiment may be connected to ICTD 100 by lead 190. In an alternative embodiment, anode 182 may be placed on, mechanically coupled to, or otherwise situated on an external surface of ICTD case 200. Anode 182 may be a lead-like structure including, for example and without limitation, a wire, a coiled wire, a flattened metallic element, or similar structure. Anode 182, which is coupled to cathode 180, may be configured to be in close proximity to cathode 180, or may be configured to be at some distance from cathode 180.

Over the lifetime of bioelectric cell 176 and bioelectric hybrid battery system 276B.3, anode 182 is slowly consumed (that is, absorbed into the patient's body) as part of the power generation process. By placing anode 182 external to case 428 and case 200, it is possible to restore the power-generating capability of bioelectric hybrid battery system 276B.3 by replacing only anode 182. For example, anode 182 may be situated in a body cavity close to a skin surface of a patient. As a result, any surgery necessary to replace anode 182 may be minimally invasive for the patient.

FIG. 4D presents a block diagram of another exemplary bioelectric hybrid battery system 276B.4 according to an embodiment of the present system and method. Many elements of exemplary bioelectric hybrid battery system 276B.4 are substantially the same or similar to those presented in conjunction with exemplary bioelectric hybrid battery systems 276B.1, 276B.2, 276B.3 discussed above (see FIGS. 4A, 4B, and 4C), and a detailed discussion of those elements will not be repeated here. In FIG. 4D, the dashed box (labeled 276B.4) contains those elements which may comprise exemplary bioelectric hybrid battery system 276B.4.

In bioelectric hybrid battery system 276B.4, electrical power (that is, current and/or voltage) from bioelectric cell 176 is used to charge secondary cell 404 via charging means 406, in a manner substantially the same or similar to that already described above in conjunction with other embodiments. In addition, power from bioelectric cell 176 may also be used to directly power some operations of ICTD 100.

In the exemplary embodiment illustrated in FIG. 4D, power from bioelectric cell 176 is delivered to an internal power coupling 422 of bioelectric hybrid battery system 276B.4. Power from secondary cell 404 is also delivered to internal power coupling 422. Bioelectric cell 176 may deliver a first power level, while secondary cell 404 may deliver a second power level. For example, bioelectric cell 176 may deliver a voltage of in a range of approximately 0.5 volts to 2 volts, and a current in a range of approximately 100 μAmps to 150 μAmps. Secondary cell 404 may deliver a voltage of approximately 4 volts and a current of approximately 3 to 5 amps. Persons skilled in the relevant arts will recognize that the voltages and currents described here are exemplary only, and other voltage and/or current levels may be delivered as well.

Both the first power level and the second power level are delivered to hybrid battery power coupling 422. The first power level and the second power level are delivered from hybrid battery power coupling 422 to battery terminal 298 of ICTD 100 via lead 190. The first power level and the second power level are delivered from battery terminal 298 to ICTD power coupling 223 via ICTD internal power line 296. From ICTD power coupling 223, either the first power level or the second power level may be delivered to various elements of ICTD 100.

For example, in an embodiment, a first power level from bioelectric cell 176 may be a low voltage, low current power from bioelectric cell 176 (for example, approximately 100 μAmps and approximately 0.5 volts up to approximately 2 volts, depending on the exact configuration of bioelectric cell 176). The first power level may be delivered to ICTD operations circuitry 430′ via a first internal power bus 294.1. ICTD operations circuitry 430′ may be similar to ICTD operations circuitry 430 already discussed above and may include elements of ICTD 100 which can be powered at low voltage and/or low current levels. Low voltage/low current ICTD operations circuitry 430′ may include, for example and without limitation, memory 260, telemetry circuit 264, physiological sensor 270, impedance measuring circuit 278, microcontroller 220, atrial pulse generator 222, atrial sensing circuits 244, ventricular sensing circuits 246, analog-to-digital converter 252, and electrode configuration switch 226.

However, ICTD low voltage/low current operations circuitry 230′ may explicitly exclude shocking circuit 282 (which was included in ICTD operations circuitry 230, discussed above). In an embodiment of the present system and method, shocking circuit 282 requires high voltages and currents, and therefore cannot be powered off of a voltage or current provided directly from bioelectric cell 176. Instead, shocking circuit 282 requires higher voltages and/or currents provided by secondary cell 404. In an alternative embodiment, some control or switching circuitry associated with or comprising shocking circuit 282 may be powered off of low voltages or low currents, and therefore may be powered via electricity provided by bioelectric cell 176. However, a shocking capacitor or shocking capacitors 424, which are used to store up high voltages prior to shocking, may still require high voltages. Therefore, a shocking capacitor or shocking capacitors 424 associated with shocking circuit 282 will still be powered by electricity from secondary cell 404.

Shown in FIG. 4D is a second power bus 294.2 which provides high voltage and/or high current to shocking circuit 282 via power coupling 223. For example, the voltage may be an unloaded voltage of approximately 4 to 4.2 volts, or a loaded voltage of approximately 3.5 volts, or a current of approximately 3 amps to 4.5 amps.

Persons skilled in the relevant arts will recognize that ICTD 100 may further comprise control circuitry used to determine power routing from power coupling 223 to elements of ICTD 100 via first and second power buses 294.1, 294.2. Such control circuitry may for example be part of microcontroller 220 (described above in conjunction with FIG. 1), and may in particular be part of battery control element 286. Such control circuitry may also be an element of bioelectric hybrid battery system 276B which is apart from microcontroller 220, but which may be coupled to microcontroller 220. Persons skilled in the relevant arts will further recognize that more than two power levels may be employed, along with possibly additional power buses 294.n (not shown in the FIGS. 4A-4D).

In the exemplary embodiment shown in FIG. 4D, a first power level from bioelectric cell 176 and a second power level from a secondary cell 404 are routed to elements of ICTD 100, where both the bioelectric cell 176 and the secondary cell 404 are elements of a bioelectric hybrid battery system 276B.4 which is wholly external to ICTD 100. The power is routed via various power couplings 422, 223 and/or power lines or buses 416.1, 416.2, 190, 296, 294.1, 294.2, as illustrated in the figure and as described in the exemplary embodiment above.

However, in alternative embodiments, a first power level and a second power level from a respective bioelectric cell 176 and a secondary cell 404 may be routed to elements of ICTD 100, even if one or both of bioelectric cell 176 and/or secondary cell 404 are partly or wholly internal to ICTD 100. Persons skilled in the relevant arts will recognize that in such alternative embodiments, suitable changes may be made in the linkages, arrangements, connections, or configurations of various power couplings 422, 223 and/or power lines or buses 416.1, 416.2, 190, 296, 294.1, 294.2, in order to achieve the requisite routing of power to elements of ICTD 100.

In an alternative embodiment of the present system and method, when power is routed from secondary cell 404 to shocking circuit 282, secondary cell 404 may be temporarily decoupled from bioelectric cell 176. For exemplary embodiments of circuitry which may decouple secondary cell 404 from a primary cell (which may be a bioelectric cell 176), see above referenced U.S. patent application Ser. No. ______, Attorney Docket Number A06E3099.

Persons skilled in the relevant arts will further appreciate that the exact configurations, connections, and arrangements of electrical components shown in FIGS. 4A-4D are exemplary only. Additional components, fewer components, alternative components, and variations in the connections may be employed consistent with the system and method for a hybrid battery system described herein.

### 9. Choice of Secondary Power Cell

Several elements distinguish the present system and method with respect to both prior batteries employed for use in ICTDs and to prior hybrid battery systems. Among these elements are the choices of power cells employed with the present system and method.

The lithium/silver vanadium oxide (Li/SVO) cell has been used as a power source of ICTDs 100 for many years. While it has some desirable electrical properties, the internal resistance for both the anode and cathode increase as a result of the discharging process, particularly during midlife. This may ultimately result in premature battery replacement.

The choice of a bioelectric cell 176 as a primary power source provides a long-term source of power which is safe, reliable, has an extended lifetime (minimizing the frequency of surgery for replacement), and provides for convenient replacement of the power source. In addition, and for as long as anode material 182 is not fully consumed, bioelectric cell 176 does not suffer the degradation in electrical properties associated with the Li/SVO cell, as described above.

The inventors have investigated the performance properties of the Li ion polymer cell for use as secondary cell 404, particularly in the context of charging shocking capacitors 424 within an ICTD. A shocking process (that is, a defibrillation process) may be a single shock, but more typically is a series of shocks closely spaced in time. For example, a series of shocks may be spaced 5 to 10 seconds apart, though shorter or longer intervals are possible. It is therapeutically preferable that the ICTD be capable of delivering multiple shocks within a few seconds of each other, with the option of spacing the shocks at intervals of 5 seconds or less.

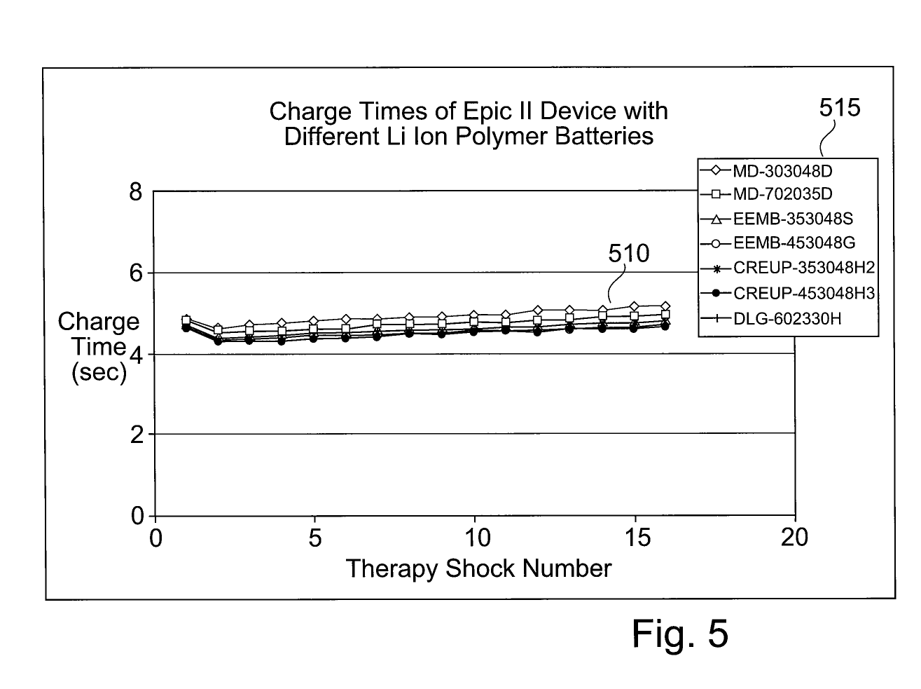

FIG. 5 shows a set of plots 510 of the measured time required, in seconds, for various Li ion polymer cells (listed in legend 515 at right) to charge the shocking capacitors to approximately 750 to 800 volts in a representative ICTD (the Epic II ICD, manufactured by St. Jude Medical, Inc., of St. Paul, Minn.). The discharge current of the Li ion polymer cells was set at approximately 3 Amperes. As can be seen from plots 510, charging times were consistently at or below approximately 5 seconds, with only a slight increase in charging times over a series of shocks.

As discussed further below in conjunction with FIG. 5, charging times of approximately 5 seconds were specifically associated with a discharge current of approximately 3 Amperes. Emerging Li ion polymer cells are capable of significantly higher currents, of approximately 4 to 4.5 Amperes, which may result in charging times of approximately 2.5 to 3 seconds, or even less.

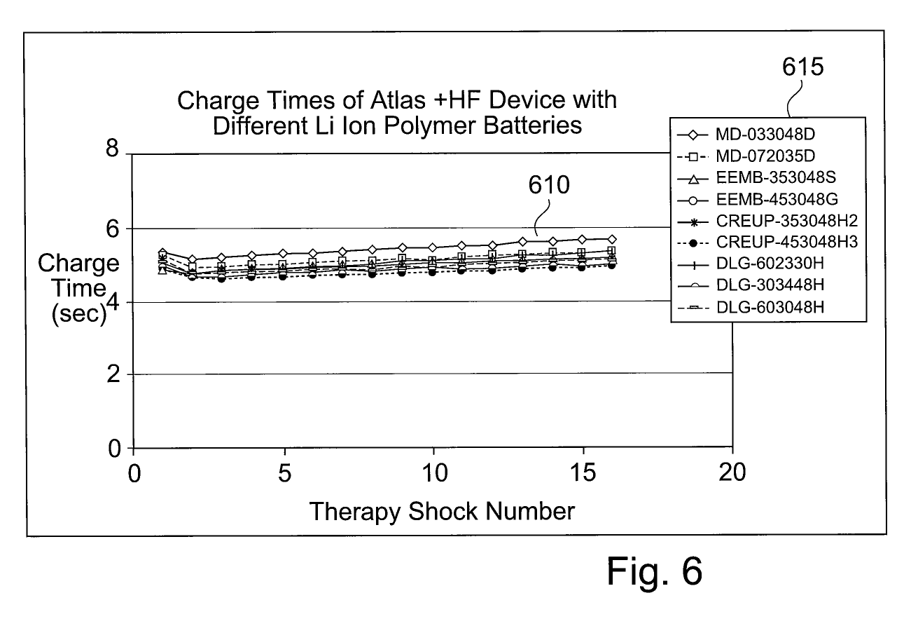

FIG. 6 shows a set of plots 610 of the time required, in seconds, for various Li ion polymer cells (listed in legend 615 at right) to charge the shocking capacitors to approximately 750 to 800 volts in another representative ICTD (the Atlas +HF ICD, manufactured by St. Jude Medical, Inc., of St. Paul, Minn.). Again, a current of approximately 3 Amperes from the Li ion polymer cells was employed. As can be seen from plots 610, charging times were consistently in the neighborhood of 5 seconds, and in many cases below 5 seconds with some of the cells tested.

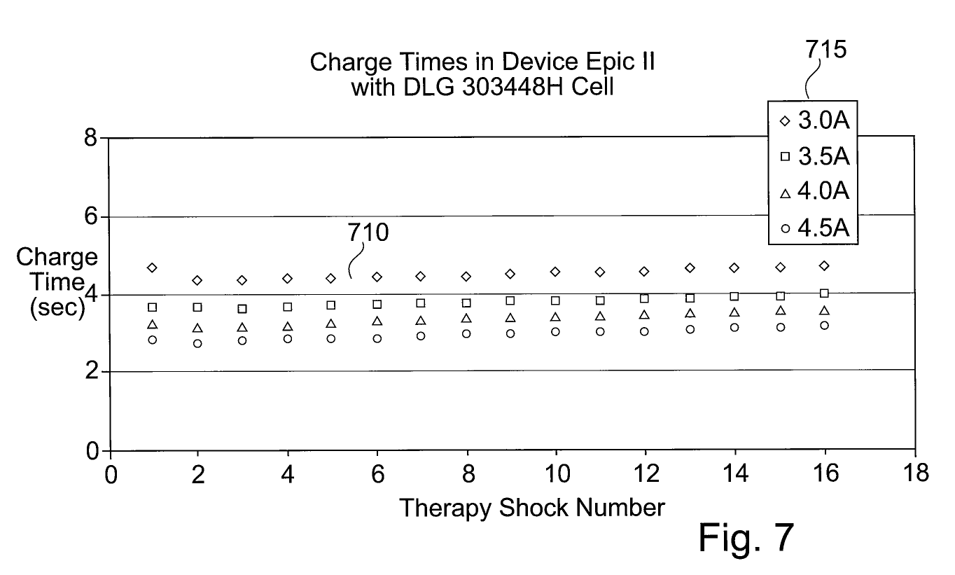

FIG. 7 shows a set of plots 710 of the time required, in seconds, for a representative Li ion polymer cell (the DLG 303448H, manufacturer DLG Battery (Shanghai) Co., Ltd., Fengxian District, Shanghai, China) to charge the shocking capacitors to approximately 750 to 800 volts in a representative ICTD (the Epic II ICD, manufactured by St. Jude Medical, Inc., of St. Paul, Minn.). Different current levels (listed in legend 715) were employed, ranging from 3 Amps to 4.5 Amps. As can be seen from plots 710, charging times of well under 5 seconds could be achieved, in some cases being lower than 2.5 seconds.

A charge time of 5 seconds or less represents a significant improvement over charge times available with present systems using Lithium Silver Vanadium Oxide (Li/SVO) batteries. Further, the Li ion polymer cell can provide current levels on the order of several Amps (for example, 3 to 5 Amps), thereby enabling the charge times on the order of 5 seconds or less, in some cases even less than 3.5 seconds, or even less than 3 seconds. Using a standard Li ion cell, current levels of 3 to 5 Amps could only be provided by a standard cell of undesirable size and weight, or a combination of multiple standard Li ion cells of undesirable size and weight, for the present application. Therefore, and as also discussed in further detail below, a Li ion polymer cell is to be preferred over a standard Li ion cell for the present system and method.

The Lithium ion polymer (Li ion polymer) cell, already described above as being used as the secondary cell 404 in exemplary embodiments of the present system and method, has both a higher voltage and lower internal resistance compared to the Li/SVO cell, making it desirable for use as the cell which charges shocking capacitor(s) 424 of ICTD 100.

In particular, the Li ion polymer cell has a higher current output than the Li/SVO cell. The discharge current of a typical Li/SVO battery used in an ICTD is approximately 3 Amps. A Li ion polymer cell may be discharged with a higher current, such as 3.5 to 4.5 Amps. Therefore, using the Li ion polymer cell as the power source 404 for the shocking capacitors 424, the discharge time, or equivalently, the time to charge the shocking capacitors 424, may be less than with the Li/SVO cell. For example, while it typically requires 10 to 18 seconds for a Li/SVO cell to charge the shocking capacitors 424 to approximately 750 volts to 800 volts, a Li ion polymer cell may charge the shocking capacitors to the same voltage (approximately 750 volts to 800 volts) in approximately 5 seconds, or even less time.

A Li ion polymer battery with, for example, LiCoO₂ cathode material, may be recharged up to 4.23V. This is about one volt higher than a new Li/SVO battery. The internal resistance of a Li ion polymer battery may be lower than 0.1Ω. In an embodiment, the output voltage of DC-to-DC converter 406 is set at approximately 4.2 volts. In this way, Li ion polymer cell 404 can be maintained at an unloaded voltage higher than 4.0 volts. A further advantage of the Li ion polymer cell is that, unlike with the Li/SVO cell, there is no significant increase in internal resistance over the life of the Li ion polymer cell. Therefore, in the discharge process the voltage drop will be less, and the loaded voltage remains higher over the life of the Li ion polymer cell as compared with the Li/SVO cell. The unloaded voltage on the Li ion polymer cell can be maintained at approximately 4.1 to 4.2 volts, while the loaded voltage, during charging of shocking capacitor(s) 424, may be maintained at approximately 3.5 volts.

As a result of all these combined advantages of the Li ion polymer cell, the discharge time for high voltage shocking (that is, the time to charge shocking capacitor(s) 424) will be significantly less compared to the discharge time using a Li/SVO battery. The time to charge the shocking capacitors is approximately 10 to 20 seconds for the Li/SVO cells presently in use. Charging times of approximately 5 seconds or even less, such as less than 4 seconds, 3.5 seconds, or even less than 3 seconds, may be achieved with the Li ion polymer cell.

### 10. Lithium Ion Polymer Cell vs. Standard Lithium Ion Cell

Li ion polymer cells also offer advantages as a secondary cell 404, as compared with standard Li ion cells that might be considered for use in the same capacity (that is, as a candidate for secondary cell 404).

Because Li ion polymer cells use gelatinous electrolyte, their self-discharge rate is relatively lower than that of a regular Li ion battery. (The self-discharge rate reflects the rate at which a cell spontaneously loses power, even with no external load or usage, due to internal chemical reactions.) The self-discharge rate of the Li ion polymer cell is in the range from 2% to 5% per month. The self-discharge rate of the standard Li ion cell is in the range of 5% to 10% per month. Because the Li ion polymer cell has a lower self-discharge rate, it will require less electrical charge from bioelectric cell 176 (as compared with the charge that would be required if the standard Li ion cell were employed as secondary cell 404). Since less charge is required from bioelectric cell 176, more power is preserved in bioelectric cell 182 or, equivalently, anode 182 of bioelectric cell 176 is consumed more slowly. This enhances the overall functional lifetime of bioelectric cell 176 and hybrid battery system 276B.

Also, and as noted above, Li ion polymer cells can be manufactured in thin, pliable shapes that offer advantages in device packaging compared with standard Li ion batteries, which have more bulk and are generally of rigid construction.

For typical shocking purposes, a desired storage of a secondary cell might be 250 milliAmpHours. This is sufficient to provide power for a series of six shocks during a defibrillation process. A standard lithium ion cell might have a discharge current capacity of 1 C to 2 C, meaning that it can only provide current at a rate equivalent to its storage capacity, or at most twice its storage capacity. For example, a standard Li ion cell with a storage capacity of 250 milliAmpHours and a discharge current of 2 C can provide at most 500 milliAmps of current. At such a current flow, it may take a minute or several minutes to charge the shocking capacitors. This is insufficient for real-world applications, so a larger cell (or additional cells) would be required.

By contrast, a Li ion polymer cell may have a discharge current capacity of anywhere from 5 C to 20 C, or even higher. At this discharge current capacity, the Li ion polymer cell may be able to discharge at a rate from 5 times to 20 times its storage capacity. Again assuming a total cell power storage of 250 milliAmpHours, a Li ion polymer cell can deliver a current from 1.25 Amps (for a 5 C cell) to 5 Amps (for a 20 C cell). It may be possible to achieve a shocking capacitor charge time of as short as 5 seconds or even less, such as approximately 3.5 seconds, 3 seconds, or even less. This is a dramatic improvement over the charge times of approximately 10 to 20 seconds achieved with presently used Li/SVO batteries. As shown in FIG. 7 (already discussed above), with some Li ion polymer cells it may be possible to charge shocking capacitor(s) 424 in times under 3 seconds, and possibly even under 2.5 seconds, which is much less than the charge times available with present devices.

### 11. Storage Capacities and Power Delivery for Cells for Different ICTD Applications

In embodiments of the present system and method, the size and capacity of the two different types of cells (bioelectric and secondary) are appropriately selected. The selection may vary depending on the type of ICTD to be powered by the bioelectric hybrid battery system 276B.

An ICTD 100 may be a pacemaker which is not configured to provide shocking (that is, ICTD 100 is not configured to provide defibrillation). Secondary cell 404 is directly connected to pacing circuits (for example, atrial pulse generator 222 and/or ventricular pulse generator 224) as pacing power supply. The capacity of the small size secondary cell 404 could be approximately 100 milliAmpHours. A capacity of 100 milliAmpHours for bioelectric hybrid battery system 276B is more than enough to maintain programming during final testing and shelf life of ICTD 100. A capacity of 100 milliAmpHours is also sufficient for 64K and RF telemetry (that is, telemetry with transmission frequencies on the order of 100 MHz). In general, higher telemetry speeds are desirable not only for faster data rates and/or increased data density, but also for higher transmission distances (for example, distances on the order of three meters for 100 MHz telemetry, as opposed to distances of only a few inches for kilohertz transmission frequencies).

By continuously charging secondary cell 404, bioelectric cell 176 can compensate for all power consumption and can maintain the secondary cell 404 at full capacity. Therefore, a combination of bioelectric cell 176 and a small size secondary cell 404 can be a power source of pacemakers.

For the pacemaker application, the small size secondary cell 404 may be a Li ion button cell such as the LIR2450 cell (capacity 120 milliAmpHours, manufactured by PowerStream Technology, 140 South Mountainway Drive, Orem Utah 84058). However, a Li ion button cell may require more complex charging circuitry to monitor or limit the charging process. In an embodiment, a Li ion polymer cell may instead be used as secondary cell 404, which may reduced the complexity of the charging circuitry, as already described above. A small Li ion polymer cell, with a capacity of, for example, about 120 to 150 milliAmpHours, can be selected as the secondary cell. For example, possible cells are the model 042025 cell (typical capacity 120 milliAmpHours) or the model 052025 cell (typical capacity 150 milliAmpHours), both manufactured by Gaston Narada International Ltd., Kwai Chung, Hong Kong.

For 64K or RF telemetry, secondary cell 404 is occasionally discharged at 1.5 milliAmps for 30 minutes or at 5 milliAmps for 30 minutes, respectively. The power of the above-listed secondary cells 404 is sufficient for these applications.

Typically, bioelectric cell 176 and small size secondary cell 404 will be combined with other elements, as described above, to create bioelectric hybrid battery system 276B. Other elements may include, for example and without limitation, charging means 406 such as a DC-to-DC converter, as already described above. For the pacemaker application, the output voltage of the DC-to-DC converter 406 may be set at for example approximately 3.7 volts. With continuous charging by the bioelectric cell 176, the voltage of secondary cell 404 can be maintained at this level.

An ICTD 100 may be configured to provide shocking (that is defibrillation therapy), as well as cardiac pacing and monitoring. For shocking applications, a larger secondary cell 404 is required. A preferred choice may be a larger size Li ion polymer cell, with the output voltage of DC-to-DC converter 406 set at, for example, approximately 4.1 volts. In an embodiment, bioelectric cell 196 is only used to charge secondary cell 404, and so compensate for the power consumption from pacing, background operations (such as sensing and communications), shocking, and self-discharge of the Li ion polymer cell 404. In an alternative embodiment, bioelectric cell 196 may directly provide some of the power for pacing and background operations, as well as recharging the Li ion polymer secondary cell 404.

The Li ion polymer cell 404 is the power source for high voltage charging. The capacity of the Li ion polymer cell 404 should be enough for lifetime high voltage charging usage. Based on statistical data, approximately 25% to 30% of ICD battery capacity is used for high voltage charging, and the other 70% of capacity is used for pacing and background operations. It is appropriate to select a Li ion polymer cell 404 with a capacity greater than 500 milliAmpHours for this application. For example, a possible cell is the DLG 603048H cell (capacity 520 milliAmpHours, manufacturer DLG Battery (Shanghai) Co., Ltd., Fengxian District, Shanghai, China).

In general, however, and whether the application is pacing only, or pacing and shocking, it is desirable to avoid a Li ion polymer battery with too small a capacity. If the size is too small, the internal resistance will be higher, and that will negatively impact the discharge rate. In addition, if a patient requires a large number of shocks in a short time, a secondary cell 404 which is too small will be unable to provide the required number of shocks.

Cardiac shocking requires more power than cardiac pacing. In addition, a secondary cell 404 used in a shocking device is partially drained during a series of shocks. Therefore, the secondary cell 404 used in a shocking device must have enough reserve capacity to continue powering ICTD 100 operations after a shocking cycle, and before the secondary cell 404 is fully recharged. Therefore, a secondary cell 404 used for cardiac shocking applications will typically be larger (that is, have greater storage capacity, and likely a larger physical volume as well) compared with a secondary cell 404 used only for pacing.

In addition, consideration must be given to the size and configuration of a bioelectric cell 176 employed is a bioelectric hybrid battery system 276B employed for shocking applications as opposed to only pacing applications. Typically, a bioelectric cell 176 puts out a voltage in the range of 0.5 to 2 volts, and a current in a range of approximately 100 microAmps to 150 microAmps. The exact values may vary depending on the specific configuration of bioelectric cell 176.

In an ICTD 100 configured for defibrillation therapy, it is desirable to recharge secondary cell 204 as quickly as possible. A relatively larger bioelectric cell 176 may provide a higher current flow, and therefore be better adapted for faster recharging of secondary cell 204. In particular, a larger surface area for anode 182 and/or cathode 180 may result in a higher current flow.

In addition, cardiac shocking places a significant power drain on a bioelectric hybrid battery system 276B, typically consuming approximately 25% to 30% of the total power consumed over the lifetime of system 276B. Therefore, a bioelectric cell 176 configured for greater overall storage capacity is better suited for cardiac shocking purposes. In the case of a bioelectric cell 176, increased storage capacity may be achieved in whole or in part by use of a larger anode element 182.

### 12. Alternative Embodiments

In an embodiment of the present system and method, each bioelectric cell 176 and/or each secondary cell 404 (for example, each lithium ion polymer cell(s)) is a self-contained battery unit, readily coupled to conventional electrical contacts in a larger system. Secondary cell 404 in particular may be of a kind which may be purchased off-the-shelf. In an alternative embodiment, elements of bioelectric cell 176 and/or secondary cell 404 may be specially tailored for integration into the bioelectric hybrid battery system 276B of the present system and method, and/or further specially tailored for integration into ICTD 100. The details of such construction, if any, are beyond the scope of this document.

In embodiments described above, the bioelectric hybrid battery system 276B employs a single bioelectric cell 176 and a single secondary cell 404. In alternative embodiments, more than one bioelectric cell 176 may be employed. In alternative embodiments, more than one secondary cell 404 may be employed.

In embodiments described above, the bioelectric hybrid battery system employs a single type of bioelectric cell 176 and a single type of secondary cell 404. In an alternative embodiment, different types of bioelectric cells 176 may be employed in combination. In an alternative embodiment, different types of secondary cells 404 may be employed in combination, which may be suitable for different types, patterns, time durations, or required power levels of ICTD activity or ICTD elements.

In an alternative embodiment, an additional, non-rechargeable cell or cells may be integrated into the system for any of several reasons. For example, an additional, non-rechargeable cell or cells may provide additional power, or may maintain charge or power in the event of a failure of either of bioelectric cell 176 or secondary cell 404. In an embodiment, such a non-rechargeable cell or cells may have a higher voltage and/or higher current output than bioelectric cell 176, but may not have as high a voltage or have as high a current as secondary cell 404. Such a non-rechargeable cell or cells may be, for example and without limitation, a lithium-silver vanadium oxide (LI/SVO) cell, a lithium-magnesium oxide (Li/MnO₂) cell, or a lithium carbon monofluoride (LiCFₓ) cell.

Suitable switching, logic, and/or coupling circuitry may be employed to select power from and/or to otherwise support the additional power cells or additional type(s) of power cells. as appropriate.

### 13. Conclusion

It is to be appreciated that the Detailed Description section, and not the Summary and Abstract sections, is intended to be used to interpret the claims. The Summary and Abstract sections may set forth one or more but not all exemplary embodiments of the present system and method as contemplated by the inventor(s), and thus, are not intended to limit the present method and system and the appended claims in any way.

Moreover, while various embodiments of the present system and method have been described above, it should be understood that they have been presented by way of example, and not limitation. It will be apparent to persons skilled in the relevant art(s) that various changes in form and detail can be made therein without departing from the spirit and scope of the present system and method. Thus, the present system and method should not be limited by any of the above described exemplary embodiments, but should be defined only in accordance with the following claims and their equivalents.

In addition, it should be understood that the figures and screen shots illustrated in the attachments, which highlight the functionality and advantages of the present system and method, are presented for example purposes only. The architecture of the present system and method is sufficiently flexible and configurable, such that it may be utilized (and arranged) in ways other than that shown in the accompanying figures. Moreover, the steps indicated in the exemplary system(s) and method(s) described above may in some cases be performed in a different order than the order described, and some steps may be added, modified, or removed, without departing from the spirit and scope of the present system and method.

Further, the purpose of the foregoing Abstract is to enable the U.S. Patent and Trademark Office and the public generally, and especially the scientists, engineers and practitioners in the art who are not familiar with patent or legal terms or phraseology, to determine quickly from a cursory inspection the nature and essence of the technical disclosure of the application. The Abstract is not intended to be limiting as to the scope of the present system and method in any way.

What is claimed is:

1. A hybrid battery system configured to power an implantable cardiac therapy device (ICTD), comprising:
a bioelectric cell;
a rechargeable secondary cell coupled to the bioelectric cell; and
charging means configured to charge the secondary cell from the bioelectric cell.

2. The hybrid battery system of claim 1, wherein the bioelectric cell is configured to generate electrical power from a replenishable substance of a patient.

3. The hybrid battery system of claim 1, wherein the secondary cell is configured to power at least one of a pacing circuit of the ICTD, a shocking circuit of the ICTD, or a background operation circuit of the ICTD.

4. The hybrid battery system of claim 1, wherein the bioelectric cell is further configured to power at least one of a pacing circuit of the ICTD or a background operation circuit of the ICTD.

5. The hybrid battery system of claim 1, wherein the secondary cell is configured to be charged via at least one of an unregulated charging process or a continuous charging process.

6. The hybrid battery system of claim 1, wherein the charging means comprises a direct-current-to-direct-current (DC-to-DC) converter.

7. The hybrid battery system of claim 1, wherein the secondary cell is configured to provide at least one of a higher voltage or a higher current than the bioelectric cell.

8. The hybrid battery system of claim 1, wherein the bioelectric cell is configured to be external to an external case of the ICTD, wherein replacement of a power source of the ICTD entails only a replacement of the bioelectric cell.

9. The hybrid battery system of claim 1, wherein a consumable anode of the bioelectric cell is configured to be external to an external case of the ICTD, wherein replacement of a power source of the ICTD entails only a supplying of a new consumable anode.

10. An implantable cardiac therapy device (ICTD) comprising:
a pacing circuit;
a background operation circuit;
a bioelectric cell;
a rechargeable secondary cell coupled to the bioelectric cell; and
a charging means coupled to the primary cell and the secondary cell, said charging means configured to charge the secondary cell from the bioelectric cell.

11. The ICTD of claim 10, wherein the bioelectric cell is configured to generate electrical power from a replenishable substance of a patient.

12. The ICTD of claim 10, wherein the secondary cell is configured to power at least one of the pacing circuit or the background operation circuit.

13. The ICTD of claim 10, wherein the bioelectric cell is configured to power at least one of the pacing circuit or the background operation circuit.

14. The ICTD of claim 10, further comprising a shocking circuit, wherein the secondary cell is configured to power the shocking circuit.

15. The ICTD of claim 10, wherein the secondary cell is configured to be charged via at least one of an unregulated charging process or a continuous charging process.

16. The ICTD of claim 10, wherein the charging means comprises a direct-current-to-direct-current (DC-to-DC) converter.

17. The ICTD of claim 10 further comprising a first power bus and a second power bus;
the first power bus configured and arranged to deliver a first voltage level from the bioelectric cell to a first circuit of the ICTD; and
the second power bus configured and arranged to deliver a second voltage level from the secondary cell to a second circuit of the ICTD.

18. The ICTD of claim 10, wherein the bioelectric cell is configured to be external to an external housing of the ICTD, wherein replacement of a power source of the ICTD entails only a replacement of the bioelectric cell.

19. The ICTD of claim 10, wherein a consumable anode of the bioelectric cell is configured to be external to an external housing of the ICTD, wherein replacement of a power source of the ICTD entails only a replacement of the consumable anode.

20. In an implantable cardiac therapy device (ICTD) comprising:
a pacing circuit;
a background operation circuit;
a bioelectric cell;
a rechargeable secondary cell coupled to the bioelectric cell; and
a charging means coupled between the bioelectric cell and the secondary cell, the charging means configured to charge the secondary cell from the bioelectric cell;
a method for powering the ICTD, comprising:
delivering power from at least one of the bioelectric cell or the secondary cell to at least one of the pacing circuit or the background operation circuit; and
charging the secondary cell from the bioelectric cell.

21. The method of claim 20, further comprising generating electrical power from the bioelectric cell by reacting an element of the bioelectric cell with a replenishable substance of a patient.

22. The method of claim 20, further comprising delivering power from the secondary cell to a shocking circuit of the ICTD.

23. The method of claim 20, further comprising delivering power from the bioelectric cell to at least one of the pacing circuit or the background operation circuit.

24. The method of claim 20, further comprising delivering power from the secondary cell to at least one of the pacing circuit or the background operation circuit.

25. The method of claim 20, wherein said step of charging the secondary cell from the bioelectric cell comprises at least one of charging the secondary cell via a continuous charging process or charging the secondary cell via an unregulated charging process.
